(19) United States
(12) Patent Application Publication
(10) Pub. No.: US 2005/0020616 A1
(43) Pub. Date: Jan. 27, 2005

(54) Aryl fused azapolycyclic compounds

(75) Inventors: Jotham W. Coe, Niantic, CT (US); Christopher J. O'Donnell, Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/889,343

(22) Filed: Jul. 12, 2004

Related U.S. Application Data
(60) Provisional application No. 60/488,764, filed on Jul. 21, 2003.

(51) Int. Cl. C07D 221/22; A61K 31/4745

(52) U.S. Cl. 514/295; 546/97

(57) ABSTRACT

This invention is directed to compounds of the formula (I): 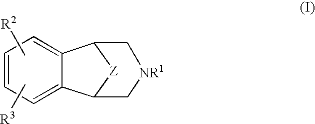
and their pharmaceutically acceptable salts, wherein R¹, R², R³ and Z are as defined herein; intermediates for the synthesis of such compounds, pharmaceutical compositions containing such compounds; and methods of using such compounds in the treatment of neurological and psychological disorders.

### BACKGROUND OF THE INVENTION

This invention relates to certain aryl fused azapolycyclic compounds defined in formula I below which bind to neuronal nicotinic acetylcholine specific receptor sites, and which are useful in modulating cholinergic function. These compounds are specifically useful in the treatment of inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrythmias, gastric acid hypersecretion, ulcers, pheochromocytoma, progressive supranuclear palsy, chemical dependencies and addictions (e.g., dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD), attention deficit disorder (ADD), restless legs syndrome (RLS), mild cognitive impairment, cognitive enhancement in schizophrenia, drug induced extrapyramidal symptoms, conduct disorder, oppositional defined disorder, anxiety in anxious smokers, cardiovascular risk in pregnancy, delayed ejaculation, emesis, symptoms due to injury inflicted by biological warfare, diarrhea, nicotine gum addiction, sleep prevention, ischemia, and Tourette's Syndrome.

The compounds of this invention may also be used in combination with an antidepressant such as, for example, a tricyclic antidepressant or a serotonin reuptake inhibiting antidepressant (SRI), in order to treat both the cognitive decline and depression associated with AD, PD, stroke, Huntington's chorea or traumatic brain injury (TBI); in combination with muscarinic agonists in order to stimulate both central muscarinic and nicotinic receptors for the treatment, for example, of ALS, cognitive dysfunction, age-related cognitive decline, AD, PD, stroke, Huntington's chorea and TBI; in combination with neurotrophic factors such as NGF in order to maximize cholinergic enhancement for the treatment, for example, of ALS, cognitive dysfunction, age-related cognitive decline, AD, PD stroke, Huntington's chorea and TBI; or in combination with agents that slow or arrest AD such as cognition enhancers, amyloid aggregation inhibitors, secretase inhibitors, tau kinase inhibitors, neuronal anti-inflammatory agents and estrogen-like therapy.

Compounds related to formula I are disclosed in J. Med. Chem., 18, 206-208 (1975) with reference to analgesic activity. Other compounds that bind to neuronal nicotinic receptor sites are referred to in WO 9818798 A1 (U.S. Pat. No. 6,235,734), WO 9935131-A1 (U.S. Pat. No. 6,410,550), U.S. Pat. No. 6,020,335 and WO9955680-A1 (U.S. Pat. No. 6,462,035). The foregoing applications are owned in common with the present application, and are incorporated herein by reference in their entirety.

### SUMMARY OF THE INVENTION

This invention relates to aryl fused azapolycyclic compounds of the formula 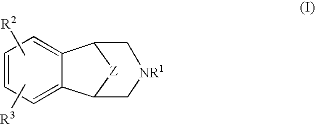
wherein Z is a group represented by the formula CR⁴R⁵ or CR⁶R⁷CR⁸R⁹;

R¹ is hydrogen, (C₁-C₆)alkyl, unconjugated (C₃-C₆)alkenyl, benzyl, XC(═O)R¹³ or —CH₂CH₂—O—(C₁-C₄)alkyl;

R² and R³ are selected, independently, from hydrogen, (C₂-C₆)alkenyl, (C₂-C₆)alkynyl, hydroxy, nitro, amino, halo, cyano, —SO_{q}(C₁-C₆)alkyl wherein q is zero, one or two, (C₁-C₆)alkylamino-, [(C₁-C₆)alkyl]₂amino-, —CO₂R¹⁰, —CONR¹¹R¹², —SO₂NR¹³R¹⁴, —C(═O)R¹⁹, —XC(═O)R¹⁹, aryl-(C₀-C₃)alkyl- or aryl-(C₀-C₃)alkyl-O—, wherein said aryl is selected from phenyl and naphthyl, heteroaryl-(C₀-C₃)alkyl- or heteroaryl-(C₀-C₃)alkyl-O—, wherein said heteroaryl is selected from five to seven membered aromatic rings containing from one to four heteroatoms selected from oxygen, nitrogen and sulfur; X²(C₀-C₆)alkyl- and X²(C₁-C₆)alkoxy-(C₀-C₆)alkyl-, wherein X² is absent or X² is (C₁-C₆)alkylamino- or [(C₁-C₆)alkyl]₂amino-, and wherein the (C₀-C₆)alkyl- or (C₁-C₆)alkoxy-(C₀-C₆)alkyl- moieties of said X²(C₀-C₆)alkyl- or X²(C₁-C₆)alkoxy-(C₀-C₆)alkyl- contains at least one carbon atom, and wherein from one to three of the carbon atoms of said (C₀-C₆)alkyl- or (C₁-C₆)alkoxy-(C₀-C₆)alkyl- moieties may optionally be replaced by an oxygen, nitrogen or sulfur atom, with the proviso that any two such heteroatoms must be separated by at least two carbon atoms, and wherein any of the alkyl moieties of said (C₀-C₆)alkyl- or (C₁-C₆)alkoxy-(C₀-C₆)alkyl- groups may be optionally substituted with from two to seven fluorine atoms, and wherein one of the carbon atoms of each of the alkyl moieties of said aryl-(C₀-C₃)alkyl- and said heteroaryl-(C₀-C₃)alkyl- may optionally be replaced by an oxygen, nitrogen or sulfur atom, and wherein each of the foregoing aryl and heteroaryl groups may optionally be substituted with one or more substituents, preferably from zero to two substituents, independently selected from (C₁-C₆)alkyl optionally substituted with from one to seven fluorine atoms, (C₁-C₆)alkoxy optionally substituted with from two to seven fluorine atoms, halo (e.g., chloro, fluoro, bromo or iodo), (C₂-C₆)alkenyl, (C₂-C₆)alkynyl, hydroxy, nitro, cyano, amino, (C₁-C₆)alkylamino-, [(C₁-C₆)alkyl]₂amino-, —CO₂R¹⁰, —CONR¹¹R¹², —SO₂NR¹³R¹⁴, —C(═O)R¹⁹ and —XC(═O)R¹⁹; with the proviso that when Z is CR⁶R⁷CR⁸R⁹ and R⁶, R⁷, R⁸ and R⁹ are all H, then R² and R³ are not both H;

or R² and R³, together with the carbons to which they are attached, form a four to seven membered monocyclic, or a ten to fourteen membered bicyclic, carbocyclic ring that can be saturated or unsaturated, wherein from one to three of the non-fused carbon atoms of said monocyclic rings, and from one to five of the carbon atoms of said bicyclic rings that are not part of the benzo ring shown in formula I, may optionally and independently be replaced by a nitrogen, oxygen or sulfur, and wherein said monocyclic and bicyclic rings may optionally be substituted with one or more substituents, preferably from zero to two substituents for the monocyclic rings and from zero to three substituents for the bicyclic rings, that are selected, independently, from (C₀-C₆)alkyl- or (C₁-C₆)alkoxy-(C₀-C₆)alkyl-, wherein the total number of carbon atoms does not exceed six and wherein any of the alkyl moieties may optionally be substituted with from one to seven fluorine atoms; nitro, oxo, cyano, halo, (C₂-C₆)alkenyl, (C₂-C₆)alkynyl, hydroxy, amino, (C₁-C₆)alkylamino-, [(C₁-C₆)alkyl]₂amino-, —CO₂R¹⁰, —CONR¹¹R¹², —SO₂NR¹³R¹⁴, —C(═O)R¹⁹, and —XC(═O)R¹⁹;

R⁴ and R⁵ are selected, independently, from H, (C₁-C₆)alkyl, F, Cl, Ph, CH₂Ph, (C₁-C₆)alkoxy, or R⁴ and R⁵, together with the carbon they are attached, form a three, four or six membered saturated ring with the proviso that R⁴ and R⁵ cannot both be H;

R⁶, R⁷, R⁸ and R⁹ are selected, independently, from H, Me, Et, Pr, Ph and CF₃;

each R¹⁰, R¹¹, R¹², R¹³, R¹⁴ and R¹⁹ is selected, independently, from hydrogen and (C₁-C₆)alkyl, or R¹¹ and R¹², or R¹³ and R¹⁴ together with the nitrogen to which they are attached, form a pyrrolidine, piperidine, morpholine, azetidine, piperazine, —N—(C₁-C₆)alkylpiperazine or thiomorpholine ring, or a thiomorpholine ring wherein the ring sulfur is replaced with a sulfoxide or sulfone; and

each X is, independently, (C₁-C₆)alkylene.

Preferably, R⁶, R⁷, R⁸ and R⁹ are H, and Z is CR⁴R⁵ where R⁴ is Me and R⁵ is H and the geometric structure may be exo or endo. Preferably, Z is CR⁴R⁵ where R⁴ is Me and R⁵ is Me. Also, preferably, R² and R³ are fluoro, nitro or trimethylfluoro.

Examples of possible heteroaryl groups within the definition of R² and R³ are the following: thienyl, oxazoyl, isoxazolyl, pyridyl, pyrimidyl, thiazolyl, tetrazolyl, isothiazolyl, triazolyl, imidazolyl, tetrazolyl, pyrrolyl and the following groups: 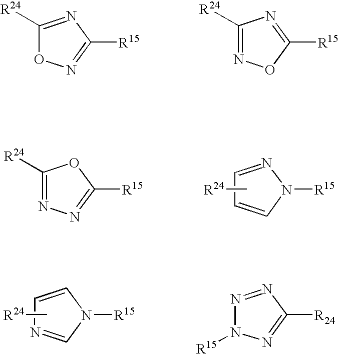
wherein one of R¹⁵ and R²⁴ is hydrogen or (C₁-C₆)alkyl, and the other is a bond to the benzo ring of formula I.

Examples of compounds of this invention are compounds of the formula I, and their pharmaceutically acceptable salts, wherein R² and R³, together with the benzo ring of formula I, form a bicyclic ring system selected from the following: 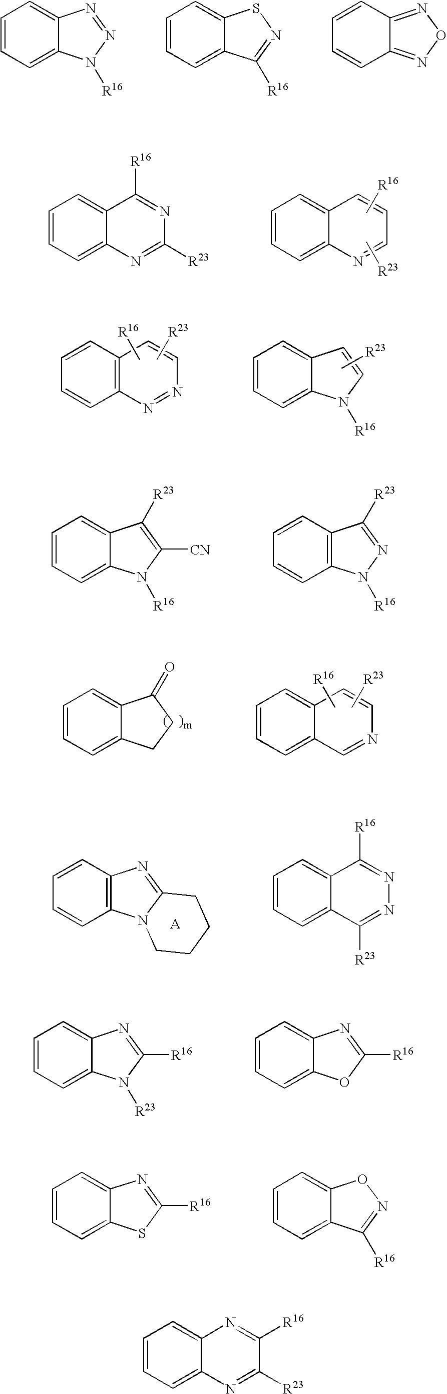
wherein R¹⁶ and R²³ are selected, independently, from hydrogen, (C₁-C₆)alkyl; and (C₁-C₆)alkoxy-(C₀-C₆)alkyl- wherein the total number of carbon atoms does not exceed six and wherein any of the alkyl moieties may optionally be substituted with from one to seven fluorine atoms; nitro, cyano, halo, amino, (C₁-C₆)alkylamino-, [(C₁-C₆)alkyl]₂amino-, —CO₂R¹⁰, —CONR¹¹R¹², —SO₂NR¹³R¹⁴, —C(═O)R¹⁹, —XC(═O)R¹⁹, phenyl and monocyclic heteroaryl wherein said heteroaryl is defined as R² and R³ are defined in the definition of compounds of the formula I above;

Other embodiments of this invention relate to compounds of the formula I, and their pharmaceutically acceptable salts, wherein R² and R³, together with the benzo ring of formula I, form a bicyclic or tricyclic ring system selected from the following:

wherein R¹⁶ and R²³ are defined as above, and m is zero, one or two, and wherein one of the carbon atoms of ring A can optionally be replaced with oxygen or N(C₁-C₆)alkyl.

Other embodiments of this invention relate to compounds of the formula I, and their pharmaceutically acceptable salts, wherein neither R² nor R³ is attached to the benzo ring of formula I via an oxygen atom.

Other embodiments of this invention relate to compounds of the formula I, and their pharmaceutically acceptable salts, wherein R² and R³ do not, together with the benzo ring of formula I, form a bicyclic or tricyclic ring system.

Other embodiments of this invention relate to compounds of the formula I wherein one or both of R² and R³ are —C(═O)R¹⁹, wherein R¹⁹ is (C₁-C₆)alkyl. Further embodiments of this invention relate to compounds of the formula I wherein one or both of R² and R³ are —C(═O)R¹⁹, wherein R¹⁹ is (C₁-C₆)alkyl or (C₁-C₃)alkyl optionally substituted with from one to seven fluorine atoms. Other embodiments relate to compounds of the formula I wherein one of R² and R³ is CF₃, fluoro, cyano, (C₂-C₆)alkynyl or C₂F₅.

The invention also relates to compounds of the formula: 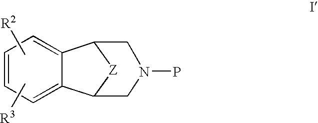
wherein R² and R³ are defined as in claim 1; and P is COOR¹⁷ wherein R¹⁷ is allyl, 2,2,2-trichloroethyl or (C₁-C₆)alkyl; —C(═O)NR¹⁰R¹¹ wherein R¹¹ and R¹² are defined as in claim 1; —C(═O)H; —C(═O)(C₁-C₆)alkyl or —C(═S)(C₁-C₆)alkyl wherein the alkyl moiety may optionally be substituted with from 1 to 3 halo atoms, preferably with from 1 to 3 fluoro or chloro atoms; benzyl, or t-butoxycarbonyl.

The present invention also relates to all radiolabeled forms of the compounds of the formula I. Preferred radiolabeled compounds of formula I are those wherein the radiolabels are selected from as ³H, ¹¹C, ¹⁴C, ¹⁸F, ¹²³I and ¹²⁵I. Such radiolabeled compounds are useful as research and diagnostic tools in metabolism studies, such as pharmacokinetics studies, etc., and in binding assays in both animals and man.

The present invention also relates to a pharmaceutical composition for use in reducing nicotine addiction or aiding in the cessation or lessening of tobacco use in a mammal, including a human, comprising an amount of a compound of the formula I, or a pharmaceutically acceptable salt thereof, that is effective in reducing nicotine addiction or aiding in the cessation or lessening of tobacco use and a pharmaceutically acceptable carrier.

The present invention also relates to a method for reducing nicotine addiction or aiding in the cessation or lessening of tobacco use in a mammal, including a human, comprising administering to said mammal an amount of a compound of the formula I, or a pharmaceutically acceptable salt thereof, that is effective in reducing nicotine addiction or aiding in the cessation or lessening of tobacco use.

The present invention also relates to a method of treating a disorder or condition selected from inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrythmias, gastric acid hypersecretion, ulcers, pheochromocytoma, progressive supranuclear palsy, chemical dependencies and addictions, dependencies on, or addictions to nicotine (or tobacco products), alcohol, benzodiazepines, barbiturates, opioids or cocaine, headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, petit mal absence epilepsy, senile dementia of the Alzheimer's type (AD), Parkinson's disease (PD), attention deficit hyperactivity disorder (ADHD), attention deficit disorder (ADD), restless legs syndrome (RLS), mild cognitive impairment, cognitive enhancement in schizophrenia, drug induced extrapyramidal symptoms, conduct disorder, oppositional defined disorder, anxiety in anxious smokers, cardiovascular risk in pregnancy, delayed ejaculation, emesis, symptoms due to injury inflicted by biological warfare, diarrhea, nicotine gum addiction, sleep prevention, ischemia, and Tourette's Syndrome in a mammal, comprising administering to a mammal in need of such treatment an amount of a compound of the formula I, or a pharmaceutically acceptable salt thereof, that is effective in treating such disorder or condition.

The present invention also relates to a pharmaceutical composition for treating a disorder or condition selected from inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrythmias, gastric acid hypersecretion, ulcers, pheochromocytoma, progressive supranuclear palsy, chemical dependencies and addictions, dependencies on, or addictions to nicotine (or tobacco products), alcohol, benzodiazepines, barbiturates, opioids or cocaine, headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, petit mal absence epilepsy, senile dementia of the Alzheimer's type (AD), Parkinson's disease (PD), attention deficit hyperactivity disorder (ADHD), attention deficit disorder (ADD), restless legs syndrome (RLS), mild cognitive impairment, cognitive enhancement in schizophrenia, drug induced extrapyramidal symptoms, conduct disorder, oppositional defined disorder, anxiety in anxious smokers, cardiovascular risk in pregnancy, delayed ejaculation, emesis, symptoms due to injury inflicted by biological warfare, diarrhea, nicotine gum addiction, sleep prevention, ischemia, and Tourette's Syndrome in a mammal, comprising an amount of a compound of the formula I, or a pharmaceutically acceptable salt thereof, and a pharmaceutically acceptable carrier.

The present invention also relates to a method for reducing nicotine addiction or aiding in the cessation or lessening of tobacco use in a mammal, comprising administering to said mammal an amount of a compound comprising an amount of a compound of the formula 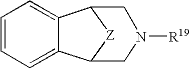
wherein R¹⁹ is selected from the group consisting of hydrogen or (C₁-C₆)alkyl and Z is as defined above, or a pharmaceutically acceptable salt thereof, that is effective in reducing nicotine addiction or aiding in the cessation or lessening of tobacco use.

The present invention also relates to a method for treating a disorder or condition selected from inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrythmias, gastric acid hypersecretion, ulcers, pheochromocytoma, progressive supranuclear palsy, chemical dependencies and addictions, dependencies on, or addictions to nicotine (or tobacco products), alcohol, benzodiazepines, barbiturates, opioids or cocaine, headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, petit mal absence epilepsy, senile dementia of the Alzheimer's type (AD), Parkinson's disease (PD), attention deficit hyperactivity disorder (ADHD), attention deficit disorder (ADD), restless legs syndrome (RLS), mild cognitive impairment, cognitive enhancement in schizophrenia, drug induced extrapyramidal symptoms, conduct disorder, oppositional defined disorder, anxiety in anxious smokers, cardiovascular risk in pregnancy, delayed ejaculation, emesis, symptoms due to injury inflicted by biological warfare, diarrhea, nicotine gum addiction, sleep prevention, ischemia, and Tourette's Syndrome in a mammal, comprising administering to a mammal in need of such treatment an amount of a compound of the formula 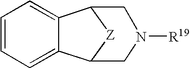
wherein R¹⁹ and Z are as defined above, or a pharmaceutically acceptable salt thereof, that is effective in treating such disorder or condition.

This invention also relates to the pharmaceutically acceptable acid addition salts of the compounds of formula I. Examples of pharmaceutically acceptable acid addition salts of the compounds of formula I are the salts of hydrochloric acid, p-toluenesulfonic acid, fumaric acid, citric acid, succinic acid, salicylic acid, oxalic acid, hydrobromic acid, phosphoric acid, methanesulfonic acid, tartaric acid, malic acid, di-p-toluoyl tartaric acid, and mandelic acid, as well salts formed from other acids known to those of skill in the art to form pharmaceutically acceptable acid addition salts to basic compounds. Other possible acid addition salts are, e.g., salts containing pharmaceutically acceptable anions, such as the hydroiodide, nitrate, sulfate or bisulfate, phosphate or acid phosphate, acetate, lactate, gluconate, saccharate, benzoate, methanesulfonate, ethanesulfonate, benzenesulfonate, and pamoate (i.e., 1,1′-methylene-bis-(2-hydroxy-3-naphthoate) salts).

Unless otherwise indicated, the term “halo”, as used herein, includes fluoro, chloro, bromo and iodo.

Unless otherwise indicated, the term “alkyl”, as used herein, includes straight chain moieties, and where the number of carbon atoms suffices, branched and cyclic moieties.

The term “alkoxy”, as used herein, means “—O-alkyl” or “alkyl-O—”, wherein “alkyl” is defined as above.

The term “alkylene, as used herein, means an alkyl radical having two available bonding sites (i.e., -alkyl-), wherein “alkyl” is defined as above.

Unless otherwise indicated, the term “one or more substituents”, as used herein, refers to from one to the maximum number of substituents possible based on the number of available bonding sites.

The compounds of formula I may have optical centers and therefore may occur in different enantiomeric configurations. The invention includes all enantiomers, diastereomers, and other stereoisomers of such compounds of formula I, as well as racemic and other mixtures thereof.

### DETAILED DESCRIPTION OF THE INVENTION

Except where otherwise stated, R¹ through R²⁵, Z, m, P and P′, and structural formula I in the reaction schemes and discussion that follow are defined as above. Schemes 1-10, below, illustrate methods of synthesizing compounds of the formula I.

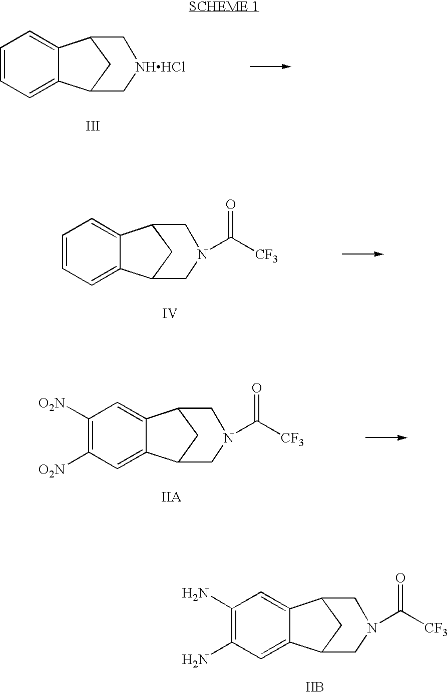

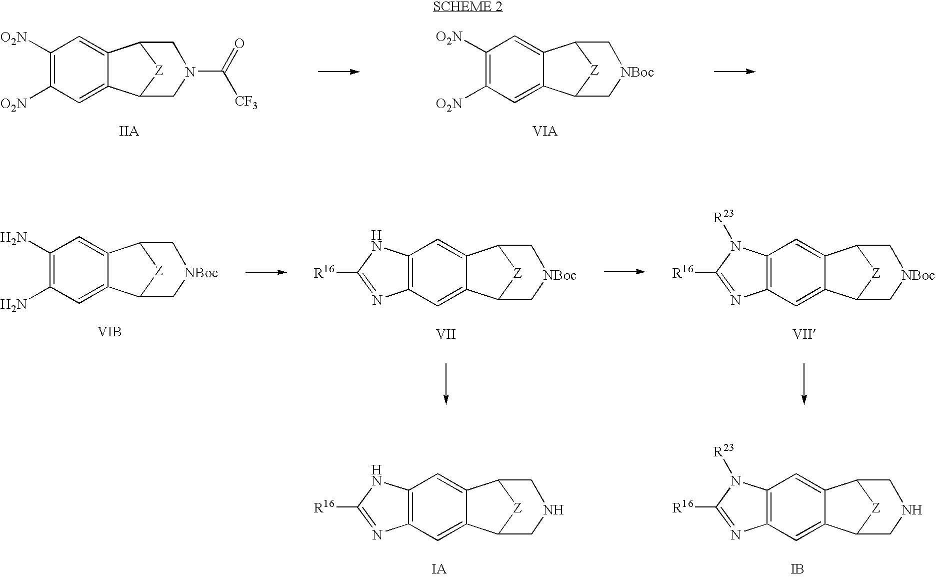

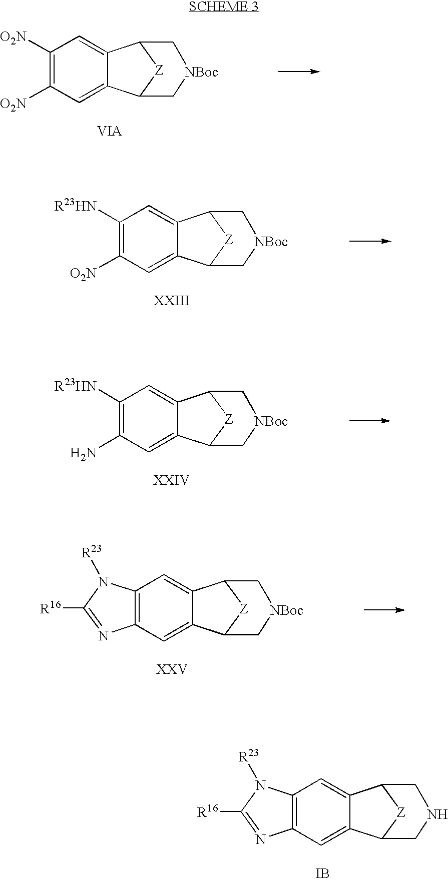

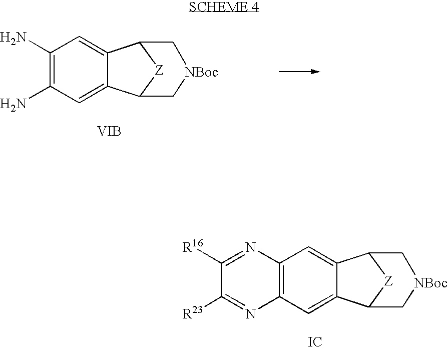

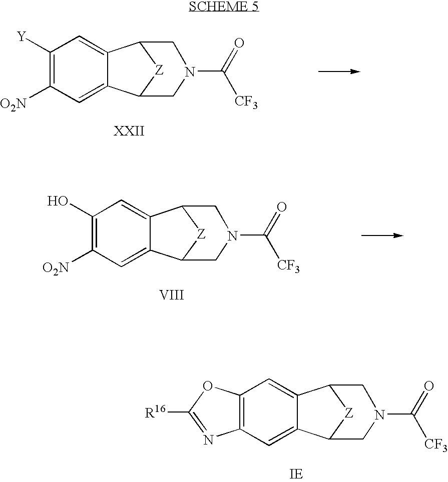

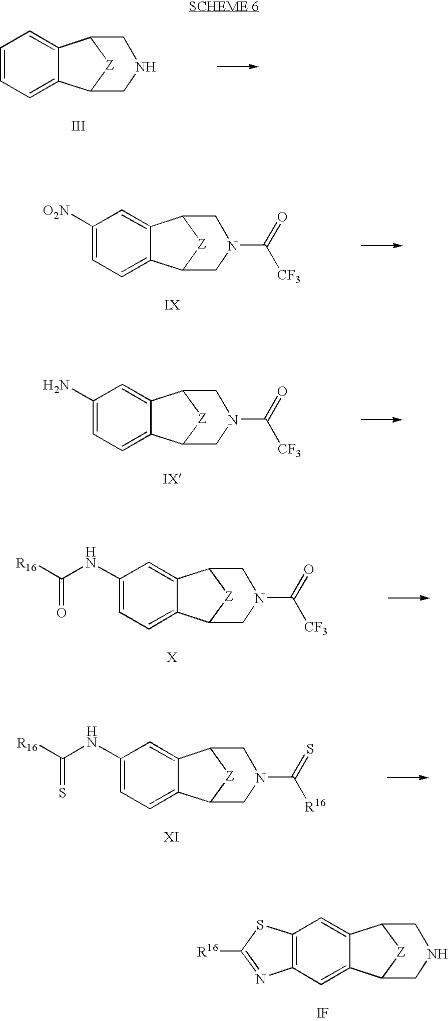

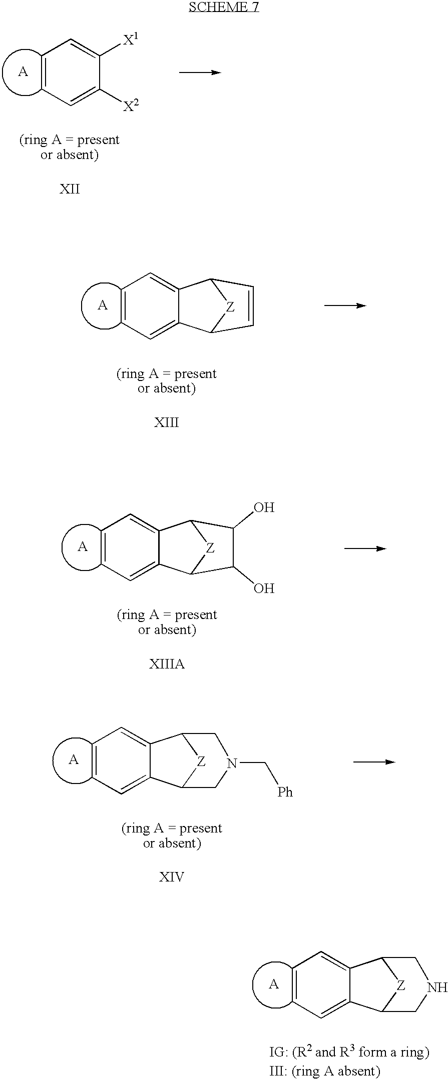

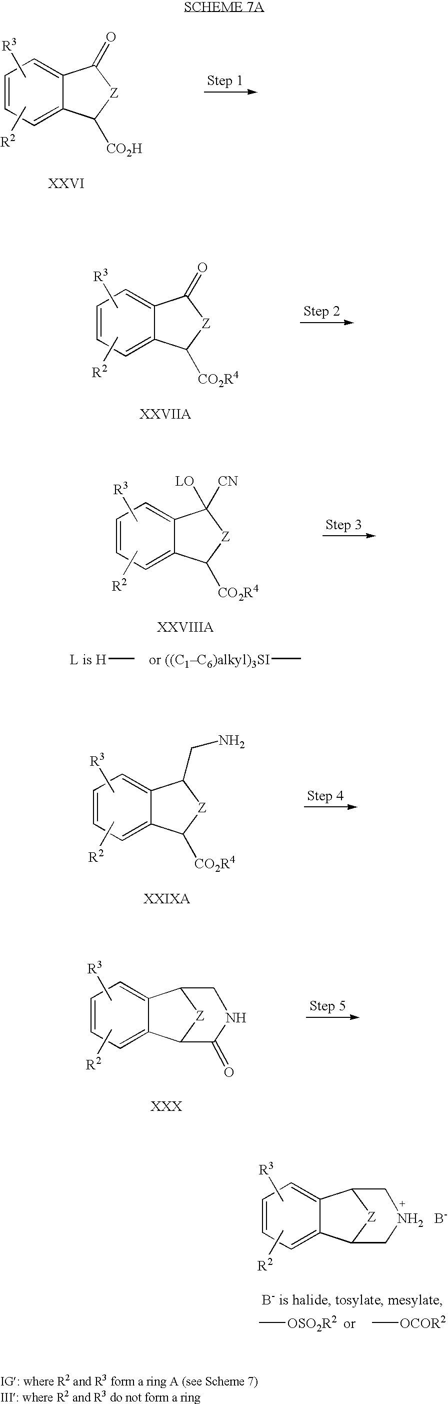

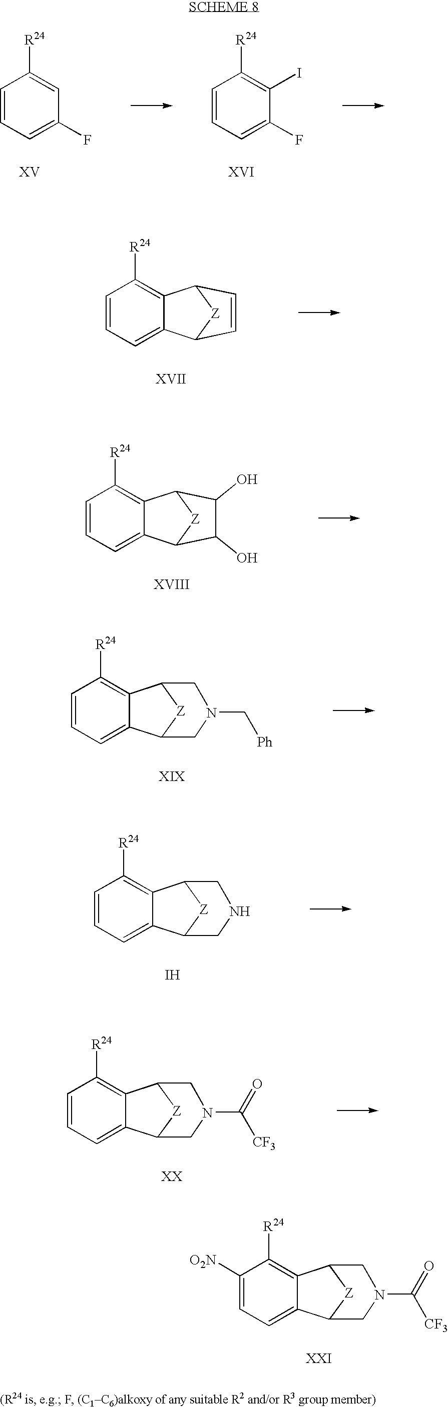

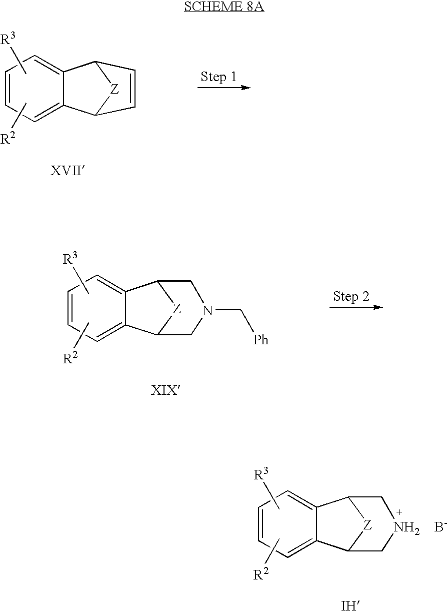

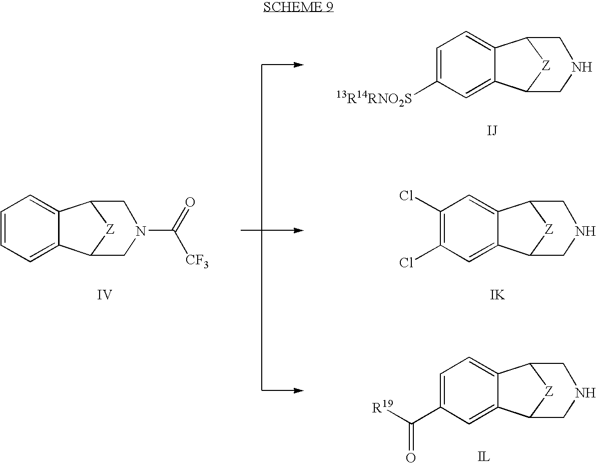

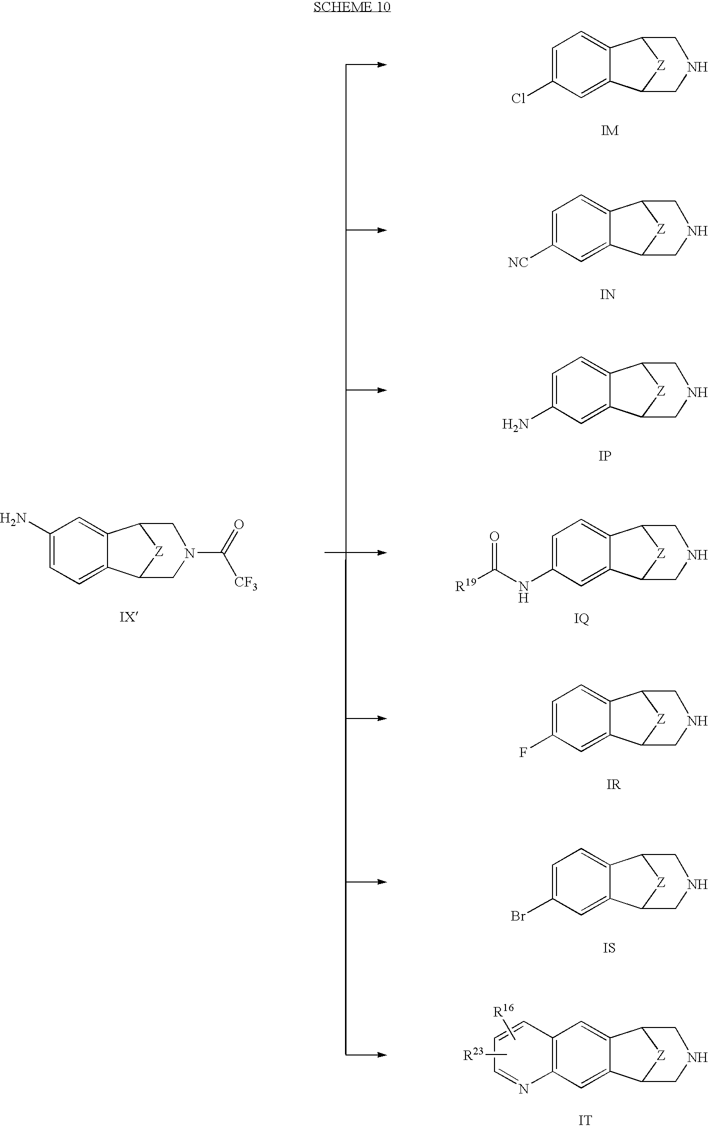

Referring to Scheme 1, the starting material of formula III is reacted with trifluoroacetic anhydride, in the presence of pyridine, to form the compound of formula IV. This reaction is typically conducted in methylene chloride at a temperature from about 0° C. to about room temperature. Other methods of generating a trifluoroacetate protecting group that may be used are recognized by those skilled in the art.

The compound of formula IV is then converted into the dinitro derivative of formula IIA by the following process. The compound of the formula IV is added to a mixture of 4 or more equivalents of trifluoromethanesulfonic acid (CF₃SO₂OH) and 2 to 3 equivalents of nitric acid, in a chlorinated hydrocarbon solvent such as chloroform, dichloroethane (DCE) or methylene chloride. The resulting mixture is allowed to react for about 5 to 24 hours. Both of the foregoing reactions are generally conducted at a temperature ranging from about −78° C. to about 0° C. for about 2 hours, and then allowed to warm to room temperature for the remaining time.

Reduction of the compound of formula IIA, using methods well known to those of skill in the art, yields the compound of formula IIB. This reduction can be accomplished, for example, using hydrogen and a palladium catalyst such as palladium hydroxide or palladium on carbon and running the reaction in an alcohol solvent, preferable methanol at about room temperature. The steps of Scheme 1 can also be performed with a nitrogen-protecting group, other than a trifluoroacetyl group, that would be deemed suitable by those of skill in the art. Other suitable nitrogen protecting groups that can be used in the procedures described throughout this document include —COCF₃, —COCCl₃, —COOCH₂CCl₃, —COO(C₁-C₆)alkyl and —COOCH₂C₆H₅. These groups may be added or removed by methods described for each in T. W. Greene and G. M. Wuts, Protective Groups in Organic Synthesis, 3^{rd} Edition (John Wiley & Sons, New York, 1999).

Referring to Scheme 2, the compound of formula IIA is converted into the corresponding compound wherein the trifluoroacetyl protecting group is replaced by a t-Boc protecting group (VIA) by reacting it first with an alkali metal or alkaline earth metal (or ammonium) hydroxide or carbonate, and then reacting the isolated product from the foregoing reaction with di-t-butyldicarbonate. Although t-Boc is used in this instance, other appropriate nitrogen-protecting groups known to those of skill in the art may be used. The reaction with the alkali or alkaline earth metal (or ammonium) hydroxide or carbonate is generally carried out in an aqueous alcohol, dioxane or tetrahydrofuran (THF) at a temperature from about room temperature to about 70° C., preferably at about 70° C., for about one to about 24 hours. The reaction of the isolated, unprotected amine or an acid addition salt of such amine, from the above reaction with di-t-butyldicarbonate is preferably carried out in a solvent such as THF, dioxane or methylene chloride at a temperature from about 0° C. to about room temperature. This reaction may or may not be conducted in the presence of a base. When the reactant is a salt of the amine, use of a base is preferred. The resulting compound of formula VIA can be converted into the corresponding diamino derivative of formula VIB using the procedure described above for converting the dinitro compound of formula IIA into the corresponding diamino compound of formula IIB, or other generally accepted nitro group reduction methods known to those of skill in the art, e.g., zinc-, tin-, or iron-mediated reductions, etc.

The conversion of the compound of formula VIB into the desired compound of the formula VII can be accomplished by reacting the compound of formula VIB with a compound of the formula XXIIA

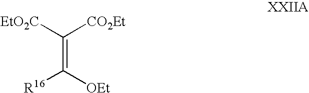

wherein R¹⁶ is hydrogen, (C₁-C₆)alkyl optionally substituted with from one to seven fluorine atoms, aryl-(C₀-C₃)alkyl wherein said aryl is selected from phenyl and naphthyl, or heteroaryl-(C₀-C₃)alkyl wherein said heteroaryl is selected from five to seven membered aromatic rings containing from one to four heteroatoms selected from oxygen, nitrogen and sulfur, and wherein each of the foregoing aryl and heteroaryl groups may optionally be substituted with one or more substituents, preferably from zero to two substituents, independently selected from (C₁-C₆)alkyl optionally substituted with from one to seven fluorine atoms, (C₁-C₆)alkoxy optionally substituted with from one to seven fluorine atoms and cyano. The preferred solvent for this reaction is a 10:1 mixture of ethanol/acetic acid. The reaction temperature can range from about 40° C. to about 100° C. It is preferably about 60° C. Other appropriate solvents include acetic acid, ethanol and isopropanol.

Alternate methods of preparing compounds of the formula VII the compound of formula VIB are described by Segelstein et al., Tetrahedron Lett., 1993, 34, 1897.

Removal of the t-Boc protecting group from the compound of formula VII yields corresponding compound of formula IA. The protecting group can be removed using methods well known to those of skill in the art. For example, the compound of formula VII can be treated with an anhydrous acid such as hydrochloric acid, hydrobromic acid, methanesulfonic acid, or trifluoroacetic acid, preferably hydrochloric acid in ethyl acetate, at a temperature from about 0° C. to about 100° C., preferably from about room temperature to about 70° C., for about one to 24 hours.

The compound of formula VII can be converted into the corresponding compound of formula IB by reacting it with a compound of the formula R²³Lg, wherein R²³ is defined as R¹⁶ is defined above, with the proviso that hydrogen is excluded from the definition of R²³, and Lg is a leaving group such as a halo or sulfonate (e.g., chloro, bromo, mesylate or tosylate), in the presence of a base such as an alkali metal hydride, hydroxide or carbonate, preferably potassium hydroxide, in a polar solvent such as water, dimethylsulfoxide (DMSO), THF or DMF, preferably a mixture of DMSO and water, and then removing the protecting group as described above. The reaction with R²³Lg is generally carried out at a temperature from about room temperature to about 100° C., preferably at about 50° C., for about five hours.

Scheme 3 illustrates an alternate method of preparing compounds of the formula IB from the compound of formula VIA. This method is the preferred method of making compounds of the formula IB wherein R²³ is a bulky group such as an aryl or heteroaryl containing group, or when R²³ can not be attached, as illustrated in Scheme 2, by alkylation or aryl substitution methods. Referring to Scheme 3, the compound of formula VIA is reacted with the appropriate compound of formula R²³NH₂ in a polar solvent such as THF, DMF or DMSO, preferably THF, at a temperature from about room temperature to about 100° C., preferably at the reflux temperature, for about four to eighteen hours. The resulting compound of formula XXIII is then converted into the corresponding compound of the formula XXIV by reducing the nitro group to an amino group using methods well known to those of skill in the art. Such methods are referred to above for the conversion of the compounds of the formula IIA into a compound of the formula IIB in Scheme 1. Closure to the imidazole ring to form the corresponding compound of formula XXV can then be accomplished by reacting the compound of formula XXIV from the above reaction with a compound of the formula XXIIA: 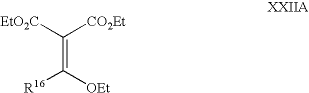
wherein R¹⁶ is defined as above, as described above for converting compounds of the formula VIB into those of the formula VII.

Removal of the protecting group from the compound of formula XXV yields the corresponding compound of formula IB. This can be accomplished using methods well known in the art, for example, as described above for forming compounds of the formula IA from the corresponding compounds of the formula VII.

Scheme 4 illustrates a method of preparing compounds of the formula IC, wherein R¹⁶ and R²³ are as defined above. Referring to Scheme 4, the compound of formula VIB, or analogously formula IIB in Scheme I, is reacted with a compound of the formula 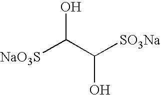
(sodium bisulfite ethane dione addition adduct) in water or another polar solvent such as THF, DMF or DMSO, preferably a mixture of water and a water miscible solvent such as THF, for about one to four hours. The reaction temperature can range from about 40° C. to about 100° C., and is preferably at about the reflux temperature.

Alternatively, the compound of formula VIB can be reacted with a compound of the formula 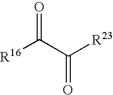
(double condensation reaction) in a polar solvent such as THF, water, or acetic acid, preferably a mixture of water and THF. This reaction is typically carried out at a temperature from about 40° C. to about 100° C., preferably at the reflux temperature, for about two to four hours. The desired quinoxoline of formula IC can then be formed by deprotecting the compound formed in either of the foregoing reactions, using the method described above for converting a compound of the formula VII into one of the formula IA. Alternatively, in place of compound VIB in Scheme 4, the compound IIB of Scheme 1 may be used analogously in this procedure with deprotection/reprotection as outlined in Scheme 2 (i.e., the process of transforming IIA to VIA) in order to arrive at ultimately the compound IC. In general, alternative nitrogen protection groups are equally suited to the procedure of Scheme 4.

Scheme 5 illustrates a method of preparing compounds of the formula I wherein R² and R³, together with the benzo ring to which they are attached, form a benzoxazole ring system. Such a compound, wherein R¹ is hydrogen, is depicted in Scheme 5 as chemical formula IE. Referring to Scheme 5, the compound of formula XXII, wherein Y is nitro, halo, trifluoromethanesulfonate or a diazonium salt, is reacted with potassium acetate or another alkali or alkaline earth metal carboxylate in a solvent such as dimethylsulfoxide (DMSO), DMF or acetonitrile, preferably DMSO. This reaction is generally allowed to run for about 12-24 hours. Appropriate reaction temperatures range from about 70° C. to about 140° C. Approximately 100° C. is preferred.

The above reaction yields the compound of formula VIII, which can then be converted into the desired compound having formula IE by the following procedure. First, the compound of formula VIII is reduced by reaction with hydrogen and a palladium or platinum catalyst such as palladium hydroxide in an alcohol solvent, preferable methanol at a temperature from about 0° C. to about 70° C., preferably at about room temperature, to form the corresponding amino derivative. The product of this reaction is then reacted with an acid chloride of the formula R¹⁶COCl or an acid anhydride of the formula (R⁶CO)₂O wherein R¹⁶ is (C₁-C₆)alkyl, or a compound of the formula R¹⁶C(OC₂H₅)₃, in an appropriate inert solvent such as decalin, chlorobenzene or xylenes. A mixture of xylenes is preferred. This reaction is typically conducted at a temperature from about 120-150° C., preferably at about 140° C. When R¹⁶COCl is used as a reactant, it is preferable to add a stoichiometric amount of triethylamine (TEA) or another organic tertiary amine base and a catalytic amount of pyridinium p-toluenesulfonic acid or pyridinium p-toluenesulfonate (PPTs) to the reaction mixture. When R¹⁶C(OC₂H₅)₃ is used as a reactant, it is preferable to add a catalytic amount of PPTs to the reaction mixture.

Removal of the trifluoroacetyl nitrogen protecting group yields the desired compound of the formula IE. This can be accomplished using methods well known to those of skill in the art, for example, reacting the protected compound with a lower alkanol and an aqueous alkali or alkaline earth metal (or ammonium) hydroxide or carbonate, aqueous sodium carbonate, at a temperature from about 50° C. to about 100° C., preferably at about 70° C., for about two to six hours.

Scheme 6 illustrates the preparation of compounds of the formula I wherein R¹ is hydrogen and R² and R³, together with the benzo ring to which they are attached, form a benzothiazole ring system. Referring to Scheme 6, the compound of formula III is reacted with trifluoroacetic anhydride to form the corresponding compound wherein the ring nitrogen is protected by a trifluoroacetyl group, and the resulting nitrogen protected compound is then reacted with two equivalents of trifluoromethanesulfonic anhydride and one equivalent of nitric acid to form the corresponding compound of formula IX, wherein there is a single nitro substituent on the benzo ring. The reaction with trifluoroacetic acid is typically conducted in the presence of pyridine. Both of the above reactions are typically conducted in a reaction inert solvent such as a chlorinated hydrocarbon solvent, preferably methylene chloride, at a temperature from about 0° C. to about room temperature, preferably at about room temperature.

The above transformation can also be accomplished using other nitration methods known to those skilled in the art. Reduction of the nitro group to an amine group can be accomplished as described above to provide a compound of the formula IX′.

The compound of formula IX′ is then reacted with a carboxylic acid halide or anhydride of the formula R¹⁶COX or (R¹⁶CO)₂O, wherein X is halo and R¹⁶ is hydrogen or (C₁-C₆)alkyl, and pyridine, TEA or another tertiary amine base, to form a compound of the formula X, which can then be converted to the desired compound having formula XI by reacting it with Lawesson's reagent:

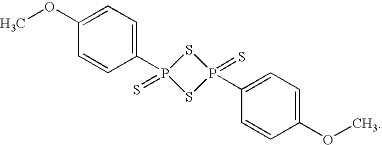

The reaction with R¹⁶COX, wherein X is halo, or (R¹⁶CO)₂O is generally carried out at a temperature from about 0° C. to about room temperature, preferably at about room temperature. The reaction with Lawesson's reagent is generally carried out in a reaction inert solvent such as benzene, 1,4-dioxane or toluene, preferably 1,4-dioxane, at a temperature from about room temperature to about the reflux temperature of the reaction mixture, preferably at about the reflux temperature.

Closure to the benzothiazole ring and nitrogen deprotection to form the desired compound of formula IF can be accomplished by reacting the compound of formula XI with potassium ferricyanide and sodium hydroxide in a mixture of water and methanol (NaOH/H₂O/CH₃OH), at a temperature from about 50° C. to about 70° C., preferably at about 60° C. for about 1.5 hours.

Scheme 7 illustrates a method of preparing the compound of formula III, which is used as the starting material for the process of Scheme 1, or a compound of the formula IG, wherein R² and R³ form a ring (labeled “A” in the Scheme), as defined above in the definition of compounds of the formula I. Referring to Scheme 7, the compound of formula XII, wherein X¹ and X² are selected, independently, from chloro, fluoro, bromo and iodo, but where at least one of X¹ and X² is Br— or I—, and reacted with cyclodiene containing a Z group as defined above, in the presence of magnesium metal, in THF, dioxane or other ethereal solvent, at a temperature from about 40° C. to about 100° C., preferably at about the reflux temperature, to form a compound of the formula XIII. Reaction of the resulting compound of formula XIII with N-methylmorpholine-N-oxide (NMO) and osmium tetroxide in acetone at about room temperature yields the corresponding compound of the formula XIIIA.

The compound having formula XIIIA is then converted into the corresponding compound of formula XIV using the following procedure. First, the compound of formula XIIIA is reacted with sodium periodate in a mixture of a chlorinated hydrocarbon, preferably dichloroethane (DCE), and water, or with lead tetraacetate in a chlorinated hydrocarbon solvent, at a temperature from about 0° C. to about room temperature, to generate a dialdehyde or glycal intermediate. The product of this reaction is then reacted with benzylamine and sodium triacetoxyborohydride in a chlorinated hydrocarbon solvent at a temperature from about 0° C. to about room temperature, preferably at about room temperature, to form the desired compound of formula XIV. Removal of the benzyl group from the compound of formula XIV yields the compound of formula III (when ring A is absent) or IG, (when ring A is present). This can be accomplished using methods well known to those of skill in the art, for example, optionally reacting the free base with one equivalent of acid, e.g., hydrochloric acid, (to form the corresponding acid addition salt), followed by hydrogenolysis and palladium hydroxide in methanol at about room temperature.

In the reductive amination step described above and throughout this document, alternatives to benzyl amine, such as ammonia, hydroxylamine, alkoxy amines, methyl amine, allyl amine, and substituted benzylamines (e.g., diphenylmethyl amine and 2- and 4-alkoxy substituted benzyl amines) can also be used. They can be used as free bases, or as their salts, preferably their acetate salts, and can be subsequently removed by methods described for each in T. W. Greene and G. M. Wuts, Protective Groups in Organic Synthesis, 3^{rd} Edition (John Wiley & Sons, New York 1999).

The procedure of Scheme 7 can also be used to prepare compounds of the formula I wherein R² and R³ do not form a ring and are not both hydrogen, by replacing the starting material of formula XII with the appropriate compound having the formula XII′

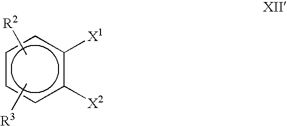

Alternatively, a compound of formula XIII can be converted, via methods described below and in Scheme 8, to compounds of formula XIV or formula IG or formula III.

An alternative means of preparing a compound of formula III′, or as appropriate IG′, is illustrated in Scheme 7A. This process can be applied to produce compounds of compounds of formula I, where R¹ is hydrogen, and R² and R³ are as defined above, with the exception of when R² and R³ are hydroxy, amino, (C₁-C₆)alkylamino, ((C₁-C₆)alkyl)₂amino, —C(═O)R¹⁹, or —(C₁-C₆)alkylene-C(═O)R¹⁹.

Referring to Scheme 7A, step 1 is an esterification of a carboxylic acid. A carboxylic acid of formula XXVI is treated with a Lewis acid catalyst such as boron trifluoride, or with an acid catalyst such as sulfuric acid, hydrochloric acid, p-toluenesulfonic acid, methane sulfonic acid, trifluoroacetic acid, or hydrobromic acid, preferably sulfuric acid, in an alcohol solvent such as methanol, ethanol, propanol, butanol, pentanol, or hexanol, preferably methanol, at a temperature between 25 and 120° C., preferably 65° C., for a period of 30 minutes to 24 hours, preferably 4 hours, to afford a compound of formula XXVIIA.

Step 2 of Scheme 7A is a cyanohydrin formation. A ketone of formula XXVIIA is treated with a Lewis acid catalyst such as zinc iodide, zinc triflate, trimethylsilyl triflate, trimethylsilyl iodide, aluminum chloride, tin (II) chloride, or trimethyl aluminum, preferably zinc iodide, or with catalytic potassium cyanide and 18-crown-6, and trimethylsilyl cyanide, in a solvent such as acetonitrile, toluene, methylene chloride, ethyl acetate, isopropyl acetate, methyl-tert-butyl ether, or tetrahydrofuran, preferably a mixture of acetonitrile and toluene, at a temperature between 0 and 100° C., preferably at 50° C., for a period of time between 1 and 24 hours, preferably 5 hours, to afford a compound of formula XXVIIIA.

Step 3 of Scheme 7A is a hydrogenolysis reaction. A nitrile of formula XXVIIIA is treated with an acid catalyst such as p-toluenesulfonic acid, methane sulfonic acid, hydrochloric acid, sulfuric acid, phosphoric acid, or trifluoroacetic acid, preferably hydrochloric acid, and a palladium catalyst such as palladium on carbon or palladium hydroxide on carbon, preferably palladium hydroxide on carbon, in a solvent such as methanol, ethanol, isopropanol, butanol, propanol, ethyl acetate, isopropyl acetate, or toluene, preferably methanol, under a hydrogen pressure of 15 to 100 psi, preferably 50 psi, for a time period between 2 and 72 hours, preferably 24 hours, to afford a compound of formula XXIXA.

Step 4 of Scheme 7A is an amide formation. An amine of formula XXIXA is treated with a base such as sodium tert-butoxide, sodium methoxide, sodium ethoxide, sodium hydroxide, potassium tert-butoxide, potassium methoxide, potassium ethoxide, potassium hydroxide, sodium carbonate, potassium carbonate, cesium carbonate, sodium hydride, triethylamine, methylimidazole, lutidine, pyridine, methylmorpholine, ethylmorpholine, or diisopropylethylamine, preferably sodium tert-butoxide, in a solvent such as methanol, ethanol, isopropanol, ethyl acetate, acetonitrile or toluene, preferably methanol, at a temperature between 0 and 120° C., preferably 65° C., for a time period between 30 minutes and 72 hours, preferably 2 hours, to afford a compound of formula XXX.

Step 5 of Scheme 7A is a reduction of an amide. An amide of formula XXX is treated with a reducing agent such as borane tetrahydrofuran complex, diborane, borane dimethylsulfide complex, lithium aluminum hydride, or a combination of sodium borohydride and boron trifluoride, preferably lithium aluminum hydride, in a solvent such as tetrahydrofuran, 1,2-dimethoxyethane, 1,2-diethoxyethane, diisopropyl ether, 1,4-dioxane, or methyl-tert-butyl ether, preferably tetrahydrofuran, at a temperature between 0 and 80° C., preferably 50° C., for time period between 1 and 24 hours, preferably 5 hours. The product is isolated by crystallization as a salt of an acid such as p-toluenesulfonic acid, methane sulfonic acid, hydrochloric acid, oxalic acid, citric acid or acetic acid, preferably p-toluenesulfonic acid, in a solvent such as isopropanol, hexane, acetone, ethyl acetate, methyl ethyl ketone, or toluene, preferably isopropanol, to afford the salt form of compound of formula IG or III.

Scheme 8, 9 and 10 illustrate methods of preparing compounds of the formula I wherein R¹ is hydrogen, and R² and R³ represent a variety of different substituents, as defined above, but do not form a ring.

Scheme 8 illustrates a variation of the process shown in Scheme 7, which can be used to make a compound identical to that of formula III except that the benzo ring is substituted with a fluoro group, an alkoxy group or any other suitable R² and/or R³ group (R²⁴ in Scheme 8). This compound is depicted in Scheme 8 as chemical structure IH. Referring to Scheme 8, where, for example, R²⁴ is F, 1,3-difluorobenzene is reacted with a strong base such as an alkali metal dialkylamine or an alkali metal alkyl (or aryl) in an ethereal solvent such as ethyl ether or THF, at a temperature below −50° C., followed by quenching with iodine or N-iodosuccinamide, to form 1,3-difluoro-2-iodobenzene. The compound 1,3-difluoro-2-iodobenzene (structural formula XVI in Scheme 8) is then converted into the compound of formula IH by a series of reactions (represented in Scheme 8 as XVI→XVII→XVIII→XIX→IH) that are analogous to the series of reactions described above and illustrated in Scheme 7 or Scheme 8A for converting compounds of the formula XIII into those of the formula IG or III. Conversion of the compound of formula XVI into the compound of formula XVII can also be accomplished by treating a mixture of the compound of formula XVI and cyclopentadiene with an alkyl lithium reagent, preferably n-butyl lithium, in an inert hydrocarbon solvent such as petroleum ether, toluene or methyl cyclohexane, at a temperature from about −20° C. to about room temperature, preferably at about 0° C. This procedure is equally effective to effect the conversion as set forth in Scheme 7 with or without the R²⁴ group present.

The compound of formula IH can then be converted into the corresponding nitrogen protected derivative of formula XX, using the methods described above for synthesizing the compound of formula IV in Scheme 1. Nitration of the compound of formula XX using the method described above for preparing the compound of formula IX in Scheme 6, yields the compound of formula XXI wherein the benzo ring is substituted with both a fluoro and nitro group, an alkoxy group and nitro group, or an R²⁴ substituent and a nitro group. The compound of formula XXI can be used to make a variety of compounds of the formula I wherein one of R² and R³ is fluoro, using methods that are well known to those of skill in the art, for example, by first converting the nitro group to an amino group, converting the amino group to a variety of other substituents, as illustrated in Scheme 10, and then removing the nitrogen protecting group.

The compound of formula XXI acts as a regioisomeric functional equivalent of the compounds having formulas IIA, VIA and XXII, in that the fluorine atom of formula XXI reacts similarly to the nitro and Y groups of formula IIA, VIA, and XXII, and thus can be subjected to the same series of reactions as those described above for the latter three compounds, providing an alternate means for preparing the products of such reactions. Similarly, the alkoxy group of formula XXI (R²⁴=alkoxy) may be converted into a hydroxyl group before or after introduction of the nitro group, and then converted to isomeric products as described above. Also, the trifluoromethanesulfonate ester of such hydroxy derivative can act as a Y-group as described.

Preparation of compounds of formula I where R²=—O(C₁-C₆)alkyl, (C₁-C₆)alkyl or aryl wherein aryl is defined as above in the definition of formula I, and R³ is H or one of the other substituents described above in the definition of formula I, can be prepared as described above and illustrated in Scheme 8 by replacing one of the fluorine atoms of the compound of formula XV with —O—(C₁-C₆)alkyl, (C₁-C₆)alkyl or aryl, respectively.

Scheme 8A illustrates an alternative procedure for obtaining compounds of formula I, where R² and R³ are as defined above, with the exception of (C₂-C₆)alkenyl, (C₂-C₆)alkynyl or nitro (IH′, as depicted). Step 1 of Scheme 8A is an oxidation followed by a reductive amination. A benzonorbornadiene derivative of formula XVII′ is first treated with ozone until the solution develops a blue color between 0° C. and −78° C., preferably −78° C., in a solvent such as methanol, or dichloromethane, preferably methanol. The ozonide formed is reduced by hydrogenolysis between −78° C. and room temperature, preferably between 0° C. and room temperature, with platinum or palladium catalyst such as platinum oxide, platinum on carbon, palladium on carbon, or palladium hydroxide on carbon, preferably 5% platinum on carbon, for a period of time between 5 minutes and 6 hours, preferably 1 hour, under a hydrogen atmosphere between 15 and 100 psi, preferably between 30 and 50 psi. Next, an arylmethylamine, such as benzylamine, 4-methoxybenzylamine, or 3,4-dimethoxybenzylamine, preferably benzylamine is added to the reaction mixture at room temperature with an acid catalyst such as formic acid, acetic acid, p-toluenesulfonic acid, oxalic acid, or hydrochloric acid, preferably formic acid, and hydrogenolysis is resumed for a period of time between 1 and 12 hours, preferably 4 hours, at a hydrogen pressure between 15 and 100 psi, preferably 50 psi, to afford a compound of formula XIX′, where Ar is an aryl group.

Step 2 of Scheme 8A is a hydrogenolysis reaction. A compound of formula II is treated with an acid such as p-toluenesulfonic acid, hydrochloric acid, sulfuric acid, acetic acid, formic acid, or methane sulfonic acid, preferably p-toluenesulfonic acid, and a palladium catalyst such as palladium hydroxide on carbon or palladium on carbon, preferably palladium hydroxide on carbon, in a solvent such as methanol, ethanol, isopropanol, ethyl acetate, or methyl acetate, preferably methanol, under a hydrogen pressure between 15 and 100 psi, preferably 50 psi, at a temperature between room temperature and 60° C., preferably 40° C., for a period of time between 1 and 48 hours, preferably 15 hours. The product is crystallized as a salt depending on which acid catalyst is used in a solvent such as isopropanol, hexane, acetone, ethyl acetate, methyl ethyl ketone, or toluene, preferably in a mixture of isopropanol and hexane, to afford a compound of formula IH′.

Scheme 9 illustrates methods of preparing compounds of the formula I wherein: (a) R¹ is hydrogen and R² is R¹³R¹⁴NO₂S—; (b) R¹ and R² are both chloro; and (c) R¹ is hydrogen and R² is R¹⁹C(═O)—. These compounds are referred to in Scheme 9, respectively, as compounds of formulas IJ, IK and IL.

Referring to Scheme 9, compounds of the formula IJ can be prepared by reacting the compound of formula IV with two or more equivalents of a halosulfonic acid, preferably chlorosulfonic acid, at a temperature from about 0° C. to about room temperature. Reaction of the chlorosulfonic acid derivative so formed with an amine having the formula R¹³R¹⁴NH, wherein R¹³ and R¹⁴ are defined as above, followed by removal of the nitrogen protecting group, yields the desired compound having formula IJ.

Compounds of the formula IK can be prepared by reacting the compound of formula IV with iodine trichloride in a chlorinated hydrocarbon solvent, followed by removal of the nitrogen protecting group. The reaction with iodine trichloride is typically carried out at a temperature from about 0° C. to about room temperature, and is preferably carried out at about room temperature. In a similar fashion, the analogous mono- or di-brominated or mono- or di-iodinated compounds can be prepared by reacting the compound of IV with N-iodosuccinamide or N-bromosuccinimide in a trifluoromethanesulfonic acid solvent, followed by removal of the nitrogen protecting group as described above.

Reaction of the compound of IV with an acid halide of the formula R¹⁹COCl or an acid anhydride of the formula (R¹⁹CO)₂O, with or without a reaction inert solvent such as a chlorinated hydrocarbon solvent, preferably methylene chloride, in the presence of Lewis acid such as aluminum chloride, at a temperature from about 0° C. to about 100° C., followed by nitrogen deprotection, yields the compound of formula IL. The reaction with the acid halide or anhydride can be carried out using other known Lewis acids or other Friedel-Crafts acylation methods that are known in the art.

The reactions described herein in which —NO₂, —SO₂NR¹³R¹⁴, —COR¹⁹, I, Br or Cl are introduced on the compound of formula IV, as depicted in Scheme 9 and described above, can be performed on any analogous compound wherein R² is hydrogen, (C₁-C₆)alkyl, halo, (C₁-C₆)alkoxy or —NHCONR¹³R¹⁴, producing compounds of the formula I wherein R² and R³ are defined as in the definition of compounds of the formula I above.

Compounds that are identical to those of the formula IL, but which retain the nitrogen protecting group, can be converted into the corresponding O-acyl substituted compounds, i.e., those wherein the —C(═O)R¹⁹ group of formula IL is replaced with a —O—C(═O)R¹⁹ group, using Baeyer-Villiger processes well known to those skilled in the art. The resulting compounds can be partially hydrolyzed to yield the corresponding hydroxy substituted compounds, and then alkylated to form the corresponding alkoxy substituted compounds. Also, such O-acyl substituted compounds can be used to prepare variably substituted benzisoxazoles.

Scheme 10 illustrates methods of making compounds of the formula I wherein: (a) R¹ is hydrogen and R² is chloro; (b) R¹ is hydrogen and R² is cyano; (c) R¹ is hydrogen and R² is amino; (d) R¹ is hydrogen and R² is R¹³C(═O)N(H)—; (e) R¹ is hydrogen and R² is fluoro; (f) R¹ is hydrogen and R² is bromo; and (g) R¹ is hydrogen and R² and R³, together with the benzo ring to which they are attached, form a quinoline ring system. These compounds are referred to in Scheme 10, respectively, as compounds of the formula IM, IN, IP, IQ, IR, IS, and IT.

Compounds of formula IM can be prepared from compounds of the formula IX′ by generation of a diazonium salt with, for instance, an alkali metal nitrite and strong mineral acid (e.g., hydrochloric acid, sulfuric acid, hydrobromic acid) in water, followed by reaction with a copper halide salt, such as copper (I) chloride. Nitrogen deprotection by the methods described above yields the desired compound of formula IM. Alternative methods for the generation of diazonium salts, as known and practiced by those of skill in the art, can also be used. The foregoing reaction is generally carried out by temperatures ranging from about 0° C. to about 60° C., preferably about 60° C. for about 15 minutes to one hour.

Reaction of the diazodium salt, prepared as described above, with potassium iodide in an aqueous medium provides the analogous iodide derivative. This reaction is generally carried out at a temperature from about 0° C. to about room temperature, preferably at about room temperature. The resulting compound, or its analogous N-tert-butylcarbonate protected form, can be used to prepare the corresponding cyano derivative by reaction with copper (I) cyanide and sodium cyanide in DMF, N,N-dimethylpropylurea (DMPU) or DMSO, preferably DMF, at a temperature from about 50° C. to about 180° C., preferably about 150° C. Nitrogen deprotection as described above provides the desired compound of formula IM.

The above described iodide derivative can also be used to access a variety of other substituents such as aryl, acetylene and vinyl substituents, as well as the corresponding carbonyl esters and amides, by palladium and nickel catalyzed processes known to those of skill in the art, such as Heck, Suzuki and Stille couplings and Heck carbonylations. These compounds and others, wherein R² is halo, alkyl, alkoxy, etc., may be similarly functionalized to generate compounds wherein R² and R³ are as defined above.

Reaction of the diazodium salt, prepared as described above, with hydrofluoric acid pyridine complex provides the analogous fluoride derivatives. This reaction is generally carried out at a temperature from about 0° C. to about 100° C., preferably at about 60° C. Nitrogen deprotection as described above provides the desired compound of formula IR.

Reaction of the diazodium salt, prepared as described above, followed by reaction with a copper halide salt, such as copper (I) bromide provides the analogous bromide derivatives. Nitrogen deprotection by the methods described above yields the desired compound of formula IS.

Nitrogen deprotection of the compound of formula IX′ provides the compound of the formula IP. The compound of formula IX′ can be reacted with a acyl group having the formula R¹⁹COCl or (R¹⁹CO)₂O using the methods described above, followed by nitrogen deprotection to provide compounds of the formula IQ. In a similar fashion, treatment of the protected amine with a compound having the formula R¹⁹SO₂X, when X is chloro or bromo, followed by nitrogen deprotection, provides the corresponding sulfonamide derivative.

Reaction of the compound of formula IX′ with glycerol in the presence of an oxidizing agent such as iodine in mineral acid, preferable sulfuric acid at a temperature between room temperature and 200° C., preferable 170° C. provides a compound of formula IT where R¹⁶ and R²³ are hydrogen. Compounds of formula IT where R¹⁶ and R²³ are as defined above can be prepared by those skilled in the art. For example, a compound of formula IT where R¹⁶ is methyl and R²³ is H can be prepared by reacting a compound of formula IX′ with crotonaldehyde in the presence of iron trichloride hexahydrate and zinc chloride. This reaction is carried out in a suitable inert reaction solvent, preferable ethanol at a temperature between room temperature and the reflux temperature of the solvent, preferable at 40° C. Removal of the nitrogen protecting group using conditions as defined above provides the desired compound of formula IT.

As noted above, suitable amine protecting groups that can be used, alternatively, in the procedures described throughout this document include —COCF₃, —COCCl₃, —COOCH₂CCl₃, —COO(C₁-C₆)alkyl and —COOCH₂C₆H₅. These groups may be removed by methods described for each in Greene, et al., Protective Groups in Organic Chemistry, referred to above. Instances where protecting groups would be modified under the reaction conditions, such as, e.g., a —COOCH₂C₆H₅ group during nitration, still permit said procedures to operate as described with said modified protecting group. Modifying the order of protecting group incorporation and/or methods of functional group introduction or modification may also be applied where appropriate.

In each of the reactions discussed above, or illustrated in Schemes 1-10, above, pressure is not critical unless otherwise indicated. Pressures from about 0.5 atmospheres to about 5 atmospheres are generally acceptable, with ambient pressure, i.e., about 1 atmosphere, being preferred as a matter of convenience.

The compounds of the formula I and their pharmaceutically acceptable salts (hereafter “the active compounds”) can be administered via either the oral, transdermal (e.g., through the use of a patch), intranasal, sublingual, rectal, parenteral or topical routes. Transdermal and oral administration are preferred. These compounds are, most desirably, administered in dosages ranging from about 0.01 mg up to about 1500 mg per day, preferably from about 0.1 to about 300 mg per day in single or divided doses, although variations will necessarily occur depending upon the weight and condition of the subject being treated and the particular route of administration chosen. However, a dosage level that is in the range of about 0.001 mg to about 10 mg per kg of body weight per day is most desirably employed. Variations may nevertheless occur depending upon the weight and condition of the persons being treated and their individual responses to said medicament, as well as on the type of pharmaceutical formulation chosen and the time period and interval during which such administration is carried out. In some instances, dosage levels below the lower limit of the aforesaid range may be more than adequate, while in other cases still larger doses may be employed without causing any harmful side effects, provided that such larger doses are first divided into several small doses for administration throughout the day.

The active compounds can be administered alone or in combination with pharmaceutically acceptable carriers or diluents by any of the several routes previously indicated. More particularly, the active compounds can be administered in a wide variety of different dosage forms, e.g., they may be combined with various pharmaceutically acceptable inert carriers in the form of tablets, capsules, transdermal patches, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents. In addition, oral pharmaceutical compositions can be suitably sweetened and/or flavored. In general, the active compounds are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tabletting purposes. Solid compositions of a similar type may also be employed as fillers in gelatin capsules; preferred materials in this connection also include lactose or milk sugar, as well as high molecular weight polyethylene glycols. When aqueous suspensions and/or elixirs are desired for oral administration the active ingredient may be combined with various sweetening or flavoring agents, coloring matter and, if so desired, emulsifying and/or suspending agents, together with such diluents as water, ethanol, propylene glycol, glycerin and various combinations thereof.

For parenteral administration, a solution of an active compound in either sesame or peanut oil or in aqueous propylene glycol can be employed. The aqueous solutions should be suitably buffered (preferably pH greater than 8), if necessary, and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

It is also possible to administer the active compounds topically and this can be done by way of creams, a patch, jellies, gels, pastes, ointments and the like, in accordance with standard pharmaceutical practice.

### Biological Assay

The effectiveness of the active compounds in suppressing nicotine binding to specific receptor sites is determined by the following procedure which is a modification of the methods of Lippiello, P. M. and Fernandes, K. G. (in The Binding of L-[³H]Nicotine To A Single Class of High-Affinity Sites in Rat Brain Membranes, Molecular Pharm., 29, 448-54, (1986)) and Anderson, D. J. and Arneric, S. P. (in Nicotinic Receptor Binding of ³H-Cystisine, ³H-Nicotine and ³H-Methylcarmbamylcholine In Rat Brain, European J. Pharm., 253, 261-67 (1994)).

### Procedure

Male Sprague-Dawley rats (200-300 g) from Charles River were housed in groups in hanging stainless steel wire cages and were maintained on a 12 hour light/dark cycle (7 a.m.-7 p.m. light period). They received standard Purina Rat Chow and water ad libitum.

The rats were killed by decapitation. Brains were removed immediately following decapitation. Membranes were prepared from brain tissue according to the methods of Lippiello and Fernandez (Mol. Pharmacol, 29, 448-454 (1986)) with some modifications. Whole brains were removed, rinsed with ice-cold buffer, and homogenized at 0° in 10 volumes of buffer (w/v) using a Brinkmann Polytron™, setting 6, for 30 seconds. The buffer consisted of 50 mM Tris HCl at a pH of 7.5 at room temperature. The homogenate was sedimented by centrifugation (10 minutes; 50,000×g; 0 to 4° C. The supernatant was poured off and the membranes were gently resuspended with the Polytron™ and centrifuged again (10 minutes; 50,000×g; 0 to 4° C.). After the second centrifugation, the membranes were resuspended in assay buffer at a concentration of 1.0 g/100 mL. The composition of the standard assay buffer was 50 mM Tris HCl, 120 mM NaCl, 5 mM KCl, 2 mM MgCl₂, 2 mM CaCl₂ and has a pH of 7.4 at room temperature.

Routine assays were performed in borosilicate glass test tubes. The assay mixture typically consisted of 0.9 mg of membrane protein in a final incubation volume of 1.0 mL. Three sets of tubes were prepared wherein the tubes in each set contained 50 μL of vehicle, blank, or test compound solution, respectively. To each tube was added 200 μL of [³H]-nicotine in assay buffer followed by 750 μL of the membrane suspension. The final concentration of nicotine in each tube was 0.9 nM. The final concentration of cytosine in the blank was 1 μM. The vehicle consisted of deionized water containing 30 μL of 1 N acetic acid per 50 mL of water. The test compounds and cytosine were dissolved in vehicle. Assays were initiated by vortexing after addition of the membrane suspension to the tube. The samples were incubated at 0 to 4° C. in an iced shaking water bath. Incubations were terminated by rapid filtration under vacuum through Whatman GF/B™ glass fiber filters using a Brandel™ multi-manifold tissue harvester. Following the initial filtration of the assay mixture, filters were washed two times with ice-cold assay buffer (5 m each). The filters were then placed in counting vials and mixed vigorously with 20 ml of Ready Safe™ (Beckman) before quantification of radioactivity. Samples were counted in a LKB Wallach Rackbeta™ liquid scintillation counter at 40-50% efficiency. All determinations were in triplicate.

### Calculations

Specific binding (C) to the membrane is the difference between total binding in the samples containing vehicle only and membrane (A) and non-specific binding in the samples containing the membrane and cytisine (B), i.e.,

Specific binding=(C)=(A)−(B).

Specific binding in the presence of the test compound (E) is the difference between the total binding in the presence of the test compound (D) and non-specific binding (B), i.e., (E)=(D)−(B).

% Inhibition=(1−((E)/(C)) times 100.

The compounds of the invention that were tested in the above assay exhibited IC₅₀ values of less than 10 μM.

The following experimental examples illustrate, but do not limit the scope of, this invention.

### EXAMPLES

The following examples illustrate the methods and compounds of the present invention. It will be understood, however, that the invention is not limited to the specific Examples. In the examples, commercial reagents were used without further purification. Purification by chromatography was done on prepacked silica columns from Biotage (Dyax Corp, Biotage Division, Charlottesville, Va.). Melting points (mp) were obtained using a Mettler Toledo FP62 melting point apparatus (Mettler-Toledo, Inc., Worthington, Ohio) with a temperature ramp rate of 10° C./min and are uncorrected. Proton nuclear magnetic resonance (¹H NMR) spectra were recorded in deuterated solvents on a Varian INOVA400 (400 MHz) spectrometer (Varian NMR Systems, Palo Alto, Calif.). Chemical shifts are reported in parts per million (ppm, δ) relative to Me₄Si (δ 0.00). Proton NMR splitting patterns are designated as singlet(s), doublet (d), triplet (t), quartet (q), quintet (quin), sextet (sex), septet (sep), multiplet (m) apparent (ap) and broad (br). Coupling constants are reported in hertz (Hz). Carbon-13 nuclear magnetic resonance (¹³C NMR) spectra were recorded on a Varian INOVA400 (100 MHz). Chemical shifts are reported in ppm (δ) relative to the central line of the 1:1:1 triplet of deuterochloroform (δ 77.00), the center line of deuteromethanol (δ 49.0) or deuterodimethylsulfoxide (δ 39.7). The number of carbon resonance's reported may not match the actual number of carbons in some molecules due to magnetically and chemically equivalent carbons and may exceed the number of actual carbons due to conformational isomers. Mass spectra (MS) were obtained using a Waters ZMD mass spectrometer using flow injection atmospheric pressure chemical ionization (APCI) (Waters Corporation, Milford, Mass.). Gas chromatography with mass detection (GCMS) were obtained using a Hewlett Packard HP 6890 series GC system with a HP 5973 mass selective detector and a HP-1 (crosslinked methyl siloxane) column (Agilent Technologies, Wilmington, Del.). HPLC spectra were recorded on a Hewlett Packard 1100 series HPLC system with a Zorbax SB-C8, 5 μm, 4.6×150 mm column (Agilent Technologies, Wilmington, Del.) at 25° C. using gradient elution. Solvent A is water, Solvent B is acetonitrile, Solvent C is 1% trifluoroacetic acid in water. A linear gradient over four minutes was used starting at 80% A, 10% B, 10% C and ending at 0% A, 90% B, 10% C. The eluent remained at 0% A, 90% B, 10% C for three minutes. A linear gradient over one minute was used to return the eluent to 80% A, 10% B, 10% C and it was held at this until the run time equaled ten minutes. Room temperature (RT) refers to 20-25° C.

### Example 1

### 10-Aza-tricyclo[6.3.2.0^{2,7}]trideca-2(7),3,5-triene

A) 4-Oxo-1,2,3,4-tetrahydro-naphthalene-1-carboxylic acid methyl ester

2-Phenylglutaric anhydride (52.2 g, 0.274 mol) and concentrated sulfuric acid (274 mL) were heated in an oil bath at 70° C. for a period of 1.5 h. The resulting mixture was allowed to cool to RT and was added to cooled solution (ice/water bath) of MeOH (550 mL) over a period of 30 min. Upon complete addition, the mixture was allowed to warm to RT and stirred for 20 h. The mixture was poured over one liter of ice. Brine (500 mL) and water (500 mL) were added and the resulting mixture was extracted with EtOAc (4×500 mL). The combined organics were washed successively with sat. NaHCO₃ (500 mL), water (500 mL) and brine (500 mL). The organics were dried (Na₂SO₄), filtered and concentrated to provide the 44.8 g (80%) of the title compound as a brown oil which was used without further purification: ¹H NMR (CDCl₃, 400 MHz) δ 8.02 (dd, 1H, J=7.9, 1.3 Hz), 7.48 (td, 1H, J=7.5, 1.3 Hz), 7.35 (td, 1H, J=7.5, 1.3 Hz), 7.29 (1H, d, J=7.9 Hz), 3.96, (t, 1H, J=5.0 Hz), 3.69 (s, 3H), 2.87 (ddd, 1H, J=17.4, 11.6, 5.0 Hz), 2.60 (dt, 1H, J=17.4, 5.0 Hz), 2.51-2.43 (m, 1H), 2.36-2.27 (m, 1H); GCMS m/z 204 (M+).

B) 4-Cyano-4-trimethylsilanyloxy-1,2,3,4-tetrahydro-naphthalene-1-carboxylic acid methyl ester

4-Oxo-1,2,3,4-tetrahydro-naphthalene-1-carboxylic acid methyl ester (28.0 g, 0.137 mol) was dissolved in CH₂Cl₂ (138 mL). ZnI₂ (0.22 g, 0.69 mmol), and I₂ (0.21 g, 0.82 mmol) were added and then TMSCN (32.95 mL, 0.247 mol) was added dropwise over 15 min. The resulting mixture was heated at reflux for 20 h. The mixture was cooled to RT and sat. NaHCO₃ (100 mL) was added, and the resulting mixture was stirred for 30 min. The mixture was partitioned and the organic layer was washed successively with sat. NaHCO₃ (100 mL), water (100 mL) and brine (100 mL). The organic layer was dried (Na₂CO₃), filtered and concentrated to afford 34.5 g (83%) of the title compound as a brown oil which was used without further purification: ¹H NMR (CDCl₃, 400 MHz) δ 7.72-7.68 (m, 1H), 7.37-7.31 (m, 2H), 7.29-7.26 (m, 1H), 3.86-3.83 (m, 1H), 3.717/3.715 (s, 3H), 2.60-2.20 (m, 4H), 0.212/0.189 (9H); GCMS m/z 303 (M+).

C) 10-Aza-tricyclo[6.3.2.0^{2,7}]triceca-2(7),3,5-triene

Pearlman's catalyst (20% Pd(OH)₂—C (50% water), 17.22 g, 12.3 mmol) was added to a solution of 4-cyano-4-trimethylsilanyloxy-1,2,3,4-tetrahydro-naphthalene-1-carboxylic acid methyl ester (24.8 g, 81.2 mmol) in MeOH (400 mL) and 3M HCl (41 mL). This mixture was shaken under an atmosphere of hydrogen (50 psi) at 50° C. for a period of 20 h. The resulting solution was filtered through a pad of Celite™ and washed with MeOH (300 mL). Sodium tert-butoxide (27.5 g, 286 mmol) was added and the resulting solution was stirred at RT for 20 h. The mixture was concentrated and the residue was dissolved in EtOAc (500 mL) and water (200 mL). The layers were partitioned and the aqueous layer was extracted with EtOAc (3×200 mL). The combined organics were washed with brine, dried (Na₂SO₄), filtered and concentrated to afford 11.2 g of 10-aza-tricyclo[6.3.2.0^{2,7}]trideca-2,4,6-trien-9-one as a white solid (GCMS m/z 187). Tetrahydrofuran (160 mL) was added to this white solid and the resulting slurry was heated in an oil bath at 45° C. A solution of LiAlH₄ in THF (1M, 120 mmol, 120 mL) was added dropwise to this mixture over a period of 60 min. The resulting mixture was heated at 45° C. for 20 h. Upon cooling to RT, a solution of water (8.65 mL) in THF (50 mL) was added dropwise to the mixture over a period of 120 min. and the resulting mixture was allowed to stir for 20 h. The solids were removed by filtration through a pad of Celite™ and the filter cake was washed with additional THF (200 mL). The filtrate was concentrated to afford 9.32 g (90%) of the title compound as an oil: ¹H NMR (CDCl₃, 400 MHz) δ 7.19 (dd, 2H, J=5.4, 3.3 Hz), 7.08 (dd, 2H, J=5.4, 3.3 Hz), 2.99-2.95 (m, 4H), 2.81-2.76 (m, 2H), 2.04-1.99 (m, 2H), 1.87-1.80 (m, 2H); ¹³C NMR (CDCl₃, 100 MHz) δ 142.2, 126.8, 126.6, 52.9, 41.9, 27.1; APCI MS m/z 174.2 (M+1).

### Example 2

### 4-Nitro-10-aza-tricyclo[6.3.2.0^{2,7}]trideca-2(7),3,5-triene

A) 1-(10-Aza-tricyclo[6.3.2.0^{2,7}]trideca-2(7),3,5-trien-10-yl)-2,2,2-trifluoro-ethanone

Trifluoroacetic anhydride (TFAA) (14.1 mL, 99.4 mmol) was slowly added to a solution of 10-aza-tricyclo[6.3.2.0^{2,7}]trideca-2(7),3,5-triene (14.8 g, 85.5 mmol) and pyridine (16.1, 199 mmol) in CH₂Cl₂ (270 mL) at 0° C. (ice bath). After ˜3 hours, the solution was poured into 0.5N aqueous HCl (100 mL) and the layers were separated. The aqueous layer was extracted with CHCl₃ (3×150 mL) and the combined organic layer was washed with 1.0N aqueous HCl (25 mL), H₂O (50 mL), saturated aqueous NaHCO₃ solution (50 mL) and brine (50 mL). This solution was dried (Na₂SO₄), filtered and concentrated. The residue was purified by chromatography, eluting with 5% EtOAC/Hexanes to afford 15.0 g (56%) of the title compound as a white solid: ¹H NMR (CDCl₃, 400 MHz) δ 7.24-7.19 (m, 2H), 7.17-7.11 (m, 2H), 4.17 (ddd, 1H, J=13.7, 5.0, 0.8 Hz), 3.87 (ddt, 1H, J=14.1, 5.0, 1.2 Hz), 3.51 (dd, 1H, J=14.1, 2.5 Hz), 3.41 (dd, 1H, J=13.7, 2.9 Hz), 3.22-3.18 (m, 2H), 2.09-1.99 (m, 2H), 1.84-1.74 (m, 2H); ¹³C NMR (CDCl₃, 100 MHz) δ 157.4, 157.1, 141.8, 141.5, 127.6, 127.3, 126.5, 126.4, 121.2, 118.3, 115.5, 112.5, 52.5, 50.3, 38.7, 38.6, 24.9, 24.8; GCMS m/z 269 (M+).

B) 1-(4-Nitro-10-aza-tricyclo[6.3.2.0^{2,7}]trideca-2(7),3,5-trien-10-yl)-2,2,2-trifluoro-ethanone

Nitric acid (0.8 mL, 12.3 mmol, 69%) was slowly added to a solution of 1-(10-aza-tricyclo[6.3.2.0^{2,7}]trideca-2(7),3,5-trien-10-yl)-2,2,2-trifluoro-ethanone (1.6 g, 3.71 mmol) in TFA (1.9 mL) at 0° C. (ice bath). The mixture was allowed to warm to RT and stirred for 4 h at which time it was poured over CHCl₃ (20 mL) and water (20 mL). The solution was neutralized with sat. NaHCO₃ (aq) and partitioned. The aqueous layer was extracted with CHCl₃ (3×20 mL). The combined organics were washed with water (20 mL) then brine (20 mL) and dried (Na₂SO₄), filtered and concentrated. The residue was purified by chromatography eluting with 15% EtOAc/Hexanes to afford 931 mg (80%) of the title compound as a glassy solid: ¹H NMR (CDCl₃, 400 MHz) δ 8.09 (ddd, 1H, J=8.3, 3.7, 2.5 Hz), 8.01 (dd, 1H, J=6.2, 2.5 Hz), 7.31 (ap t, 2H, J=8.7 Hz), 4.18-4.07 (m, 1H), 3.91-3.85 (m, 1H), 3.57(dd, 1H, J=14.1, 2.5 Hz), 3.53-3.45 (m, 1H), 3.38-3.34 (m, 2H), 2.15-2.05 (m, 2H), 1.86-1.75 (m, 2H); GCMS m/z 314 (M+).

C) 4-Nitro-10-azatricyclo[6.3.2.0^{2,7}]trideca-2(7),3,5-triene hydrochloride

1-(4-Nitro-10-aza-tricyclo[6.3.2.0^{2,7}]trideca-2(7),3,5-trien-10-yl)-2,2,2-trifluoro-ethanone (90 mg, 0.29 mmol) was stirred with Na₂CO₃ (61 mg, 0.57 mmol) in methanol (1.5 mL) and H₂O (0.5 mL) at 70° C. for 18 hours. The mixture was concentrated, water was added and the product was extracted with CH₂Cl₂. The organic layer was extracted with 1N aqueous HCl (3×20 mL) and the acidic layer washed with CH₂Cl₂ (2×20 mL). The aqueous layer was basified to pH ˜10 with Na₂CO₃(s) and product was extracted with CH₂Cl₂ (3×30 mL). The organic layer was dried (Na₂SO₄), filtered and concentrated to afford 53 mg of the title compound as the free base. This was dissolved in methanol and treated with 1N HCl in methanol, concentrated to solids to afford 41 mg (66%) of the title compound as an orange solid (mp=252° C.). Free Base: ¹³C NMR (CDCl₃, 100 MHz) δ 150.8, 147.0, 144.3, 127.2, 122.3, 121.4, 52.3, 52.2, 41.9, 41.8, 26.0; GCMS m/z 218 (M+).

### Example 3

### 6-Methyl-5-thia-7,13-diazatetrecyclo[9.3.2.0^{2,10}.0^{4,8}]hexadeca-2(10),3,6,8-tetraene Hydrochloride

A) 1-(4-Amino-10-aza-tricyclo[6.3.2.0^{2,7}]trideca-2(7),3,5-trien-10-yl)-2,2,2-trifluoro-ethanone

Hydrogenation of 1-(4-nitro-10-aza-tricyclo[6.3.2.0^{2,7}]trideca-2(7),3,5-trien-10-yl)-2,2,2-trifluoro-ethanone (3.4 g, 10.8 mmol) under a H₂ atmosphere (50 psi) and 10% Pd/C (3.44 g) in ethanol (100 mL) over 15 hours, followed by filtration through Celite™ and concentration affords 2.84 g of the title compound as a yellow oil: ¹H NMR (CDCl₃, 400 MHz) δ 7.01-6.97 (m, 1H), 6.81-6.75 (m, 2H), 5.21 (br s, 1H), 4.22-4.09 (m, 1H), 3.88-3.79 (m, 1H), 3.48-3.27 (m, 2H), 3.15-3.09 (m, 2H), 2.04-1.91 (m, 2H), 1.75-1.73 (m, 2H); GCMS m/z 284 (M+).

B) N-(10-Trifluoroacetyl-10-aza-tricyclo[6.3.2.0^{2,7}]trideca-2(7),3,5-trien-4-yl)-acetamide

1-(4-Amino-10-aza-tricyclo[6.3.2.0^{2,7}]trideca-2(7),3,5-trien-10-yl)-2,2,2-trifluoro-ethanone (637 mg, 2.24 mmol) was stirred in CH₂Cl₂ (20 mL) and treated with triethyl amine (0.37 mL, 2.7 mmol) and acetyl chloride (0.16 mL, 2.24 mmol) then stirred 18 hours at RT. Standard NaHCO₃ work-up provided the 730 mg (100%) of the title compound as a yellow oil which was used without further purification: GCMS m/z 326 (M+).

C) N-(10-Trifluorothioacetyl-10-aza-tricyclo[6.3.2.0^{2,7}]trideca-2(7),3,5-trien-4-yl)-thioacetamide

N-(10-Trifluoroacetyl-10-aza-tricyclo[6.3.2.0^{2,7}]trideca-2(7),3,5-trien-4-yl)-acetamide (730 mg, 2.24 mmol) and 2,4-bis(4-methoxyphenyl)-1,3-dithia-2,4-diphosphetane-2,4-disulfide (Lawesson's reagent) (1.81 g, 4.47 mmol) were combined in 1,2-dimethoxyethane (19 mL) and heated to 90° C. for 15 h. After cooling the reaction was concentrated and the residue was purified by chromatography (gradient elution with 6:1, then 4:1, then 2:1 Hexanes/EtOAc) to afford 594 mg (74% over two steps) of the title compound as an oil: GCMS m/z 358 (M+).

D) 6-Methyl-5-thia-7,13-diazatetracyclo[9.3.2.0^{2,10}.0^{4,8}]hexadeca-2(10),3,6,8-tetraene hydrochloride

The above oil, 2,2,2-trifluoro-N-(10-trifluorothioacetyl-10-aza-tricyclo[6.3.2.0^{2,7}]trideca-2(7),3,5-trien-4-yl)-thioacetamide, (594 mg, 1.65 mmol) was dissolved in methanol (15 mL) and 1N NaOH (12 mL) and added to potassium ferricyanide (K₃Fe(CN)₆)(2.73 g, 8.3 mmol) in H₂O (24 mL). This mixture was heated at reflux for 15 hours, cooled, concentrated and worked up with ethyl acetate/H₂O. Purification by chromatography (gradient elution with 7.5:1 CH₂Cl₂ saturated with NH₃ to 3:1 CH₂Cl₂ saturated with NH₃) afforded 33 mg (8%) of the title compound as its free base as a white solid: ¹H NMR (CDCl₃, 400 MHz) δ 7.65 (s, 1H), 7.53 (s, 1H), 3.15 (s, 1H), 3.11 (s, 1H), 3.05-2.96 (m, 4H), 2.80 (s, 3H), 2.14 (ap d, 2H, J=9.1 Hz), 1.85 (ap dd, 2H, J=10.8, 2.1 Hz); ¹³C NMR (CDCl₃, 100 MHz) δ 166.5, 152.7, 140.4, 139.3, 134.1, 120.1, 119.0, 52.5, 41.0, 26.4, 26.3, 20.3; GCMS m/z 244 (M+).

The above product was dissolved in acetone (10 mL) and treated with 2N HCl/ether (0.116 mL) and the resulting white solids were collected by filtration to afford the title compound.

### Example 4

### 5,14-Diazatetrecyclo[10.3.2.0^{2,11}.0^{4,9}]heptadeca-2(11),3,5,7,9-pentaene Hydrochloride

1-(4-Amino-10-aza-tricyclo[6.3.2.0^{2,7}]trideca-2(7),3,5-trien-10-yl)-2,2,2-trifluoro-ethanone (450 mg, 1.58 mmol), glycerol (0.69 mL, 9.48 mmol), iodine (30 mg, 0.12 mmol) and concentrated sulfuric acid (1.0 mL, 19 mmol) were combined and heated at 170° C. for 1 h. Upon cooling to RT, the mixture was poured onto ice and the pH was adjusted to pH 10 with 1 N NaOH. This mixture was extracted with CHCl₃ (5×10 mL) and the combined organic layers were washed with brine (20 mL), dried (Na₂SO₄), filtered and concentrated. Purification by chromatography (gradient elution with 10% MeOH/CHCl₃ sat. with NH₃ to 35% MeOH/CHCl₃ sat. with NH₃) afforded 195 mg (55%) of the title compound as it's free base. This material was treated with 1N HCl in MeOH (2.17 mL) and concentrated to a white solid. Recrystallization from MeOH/Et₂O afforded the title compound as a solid: ¹H NMR (CD₃OD, 400 MHz) δ 9.21-9.17 (M, 2H), 8.24 (s, 1H), 8.15 (s, 1H), 8.09 (dd, 1H, J=8.5, 5.6 Hz), 3.77 (br s, 1H), 3.73 (br s, 1H), 3.61-3.56 (m, 2H), 3.45-3.38 (m, 2H), 2.40-2.36 (m, 2H), 2.02 (br d, 2H, J=11.2 Hz); APCI MS m/z 225.2 (M+1).

### Example 5

### 6-Methyl-5,14-diazatetracyclo[10.3.2.0^{2,11}.0^{4,9}]heptadeca-2(11),3,5,7,9-pentaene Hydrochloride

A) 1-(6-Methyl-5,14-diazatetracyclo[10.3.2.0^{2,11}.0^{4,9}]heptadeca-2(11),3.5,7,9-pentaene)-2,2,2-trifluoro-ethanone

Crotonaldehyde (190 mg, 2.38 mmol), FeCl₃.6H₂O (642 mg, 2.38 mmol) and ZnCl₂ (21 mg, 0.16 mmol) were added to a solution of 1-(4-amino-10-aza-tricyclo[6.3.2.0^{2,7}]trideca-2(7),3,5-trien-10-yl)-2,2,2-trifluoro-ethanone (450 mg, 1.58 mmol) in EtOH (6 mL) and the mixture was heated at 40° C. for 15 h. The mixture was concentrated and partitioned between ethyl acetate (10 mL) and water (10 mL). The aqueous phase was extracted with EtOAc (3×10 mL). The combined extracts were washed with brine, dried (Na₂SO₄), filtered and concentrated. The residue was purified by chromatography to afford the 85 mg (16%) of the title compound as an oil: ¹H NMR (CDCl₃, 400 MHz) δ 8.08 (d, 1H, J=8.3 Hz), 7.93 (d, 1H, J=2.9 Hz), 7.54 (d, 1H, J=10.8 Hz), 7.30 (dd, 1H, J=8.3, 1.7 Hz), 4.43-4.27 (m, 1H), 4.04-3.96 (m, 1H), 3.60-3.33 (m, 4H), 2.79 (s, 3H), 2.16-2.06 (m, 2H), 1.87-1.81 (m, 2H); GCMS m/z 334 (M+).

B) 6-Methyl-5,14-diazatetracyclo[10.3.2.0^{2,11}.0^{4,9}]heptadeca-2(11),3,5,7,9-pentaene hydrochloride

The title compound was prepared from 1-(6-methyl-5,14-diazatetracyclo[10.3.2.0^{2,11}.0^{4,9}]heptadeca-2(11),3,5,7,9-pentaene)-2,2,2-trifluoro-ethanone by the method as described in Example 2C to afford 53 mg of a white solid: ¹H NMR (CDCl₃, 400 MHz) δ 7.96 (d, 1H, J=8.3 Hz), 7.70 (s, 1H), 7.43 (s, 1H), 7.20 (d, 1H, J=8.3 Hz), 3.18-3.12 (m, 2H), 3.07-2.92 (m, 4H), 2.71 (s, 3H), 2.13-2.09 (m, 2H), 1.93-1.85 (m, 2H); ¹³C NMR (CDCl₃, 100 MHz) δ 158.3, 147.8, 145.3, 141.2, 135.8, 125.9, 125.2, 123.9, 121.5, 53.7, 53.6, 42.3, 41.8, 26.6, 26.4, 25.5; GCMS m/z 238 (M+).

### Example 6

### 4-Fluoro-10-aza-tricyclo[6.3.2.0^{2,7}]trideca-2,4,6-triene Hydrochloride

A) 1-(4-Fluoro-10-aza-tricyclo[6.3.2.0^{2,7}]trideca-2(7),3,5-trien-10-yl)-2,2,2-trifluoro-ethanone

1-(4-Amino-10-aza-tricyclo[6.3.2.0^{2,7}]trideca-2(7),3,5-trien-10-yl)-2,2,2-trifluoro-ethanone (476 mg, 1.67 mmol) was dissolved in HF-pyridine (70%, 2.05 mL, 151 mmol) and cooled to −78° C. Sodium nitrite (127 mg, 1.84 mmol) was added and the mixture was allowed to warm to RT, then heated to 60° C. for 1 h (gas evolution). After cooling to RT, water (30 mL) and CHCl₃ (75 mL) were added and the aqueous layer was neutralized with solid NaHCO₃. This mixture was filtered through Celite™ to remove all solids and partitioned. The aqueous layer was extracted with CHCl₃ (3×30 mL) and the combined organic extracts were washed with brine, dried (NaSO₄), filtered and concentrated. The crude residue was purified by chromatography (eluting with 10% EtOAc/Hexanes) to afford 230 mg (48%) of the title compound as a yellow oil: ¹H NMR (CDCl₃, 400 MHz) δ 7.10-7.04 (m, 1H), 6.89-6.82 (m, 2H), 4.08-4.03 (m, 1H), 3.83-3.77 (m, 1H), 3.56-3.42 (m, 2H), 3.21-3.14 (m, 2H), 2.06-1.96 (m, 2H), 1.80-1.73 (m, 2H); GCMS m/z 287 (M+).

B) 4-Fluoro-10-aza-tricyclo[6.3.2.0^{2,7}]trideca-2,4,6-triene hydrochloride

The title compound was prepared from 1-(4-fluoro-10-aza-tricyclo[6.3.2.0^{2,7}]trideca-2(7),3,5-trien-10-yl)-2,2,2-trifluoro-ethanone (220 mg, 0.77 mmol) by the method as described in Example 2C to afford 140 mg of a white solid. Data for free base: ¹H NMR (CDCl₃, 400 MHz) δ 7.02-6.99 (m, 1H), 6.86-6.78 (m, 2H), 2.98-2.89 (m, 4H), 2.81-2.75 (m, 2H), 2.03-1.98 (m, 2H), 1.79-1.76 (m, 2H), 1.58 (br s, 1H); ¹³C NMR (CDCl₃, 100 MHz) δ 163.1, 160.6, 144.4, 144.3, 137.9, 127.7, 127.6, 113.7, 113.5, 113.0, 112.8, 52.9, 52.7, 42.1, 41.2, 27.0, 26.6; GCMS m/z 191 (M+).

### Example 7

### 4-Chloro-10-azatricyclo[6.3.2.0^{2,7}]trideca-2,4,6-triene Hydrochloride

A) 1-(4-Chloro-10-aza-tricyclo[6.3.2.0^{2,7}]trideca-2(7),3,5-trien-10-yl)-2,2,2-trifluoro-ethanone

Copper(I)chloride (CuCl) was prepared as follows: CuSO₄ (4.3 g) and NaCl (1.2 g) were dissolved in hot H₂O (14 mL). sodium bisulfite (NaHSO₃) (1 g) and sodium hydroxide (NaOH) (690 mg) were dissolved in H₂O (7 mL) and added to the hot acidic solution over 5 minutes. The precipitated white solids were filtered and washed with water.

1-(4-Amino-10-aza-tricyclo[6.3.2.0^{2,7}]trideca-2(7),3,5-trien-10-yl)-2,2,2-trifluoro-ethanone (200 mg, 0.7 mmol) was dissolved in H₂O (1.2 mL) and concentrated HCl solution (1.2 mL) then cooled to 0° C. and treated with a solution of sodium nitrite (NaNO₂) (97 mg, 1.41 mmol) in H₂O (0.5 mL) dropwise. To the resulting solution was added a CuCl (147 mg, prepared as described above, 1.48 mmol) in concentrated HCl solution (0.6 mL) over 10 minutes (gas evolution observed). The resulting solution was warmed to 60° C. for 90 minutes, then was cooled to room temperature, diluted with water (20 mL) and extracted with CHCl₃ (4×30 mL). The combined organic extracts were washed with sat. NaHCO₃ then brine and dried (Na₂SO₄), filtered and concentrated. The residue was purified by chromatography (elutinq with 10% EtOAc/hexanes) to afford 80 mg of the title compound as an oil: ¹H NMR (CDCl₃, 400 MHz) δ 7.19-7.11 (m, 2H), 7.08-7.04 (m, 1H), 4.18-4.12 (m, 1H), 3.87-3.82 (m, 1H), 3.52-3.46 (m, 1H), 3.43-3.35 (m, 1H), 3.21-3.14 (m, 2H), 2.08-1.97 (m, 2H), 1.80-1.72 (m, 2H); GCMS m/z 303 (M+).

B) 4-Chloro-10-azatricyclo[6.3.2.0^{2,7}]dodeca-2(7),3,5-triene hydrochloride

The title compound was prepared from 1-(4-chloro-10-aza-tricyclo[6.3.2.0^{2,7}]trideca-2(7),3,5-trien-10-yl)-2,2,2-trifluoro-ethanone (76 mg, 0.25 mmol) by the method as described in Example 2C to afford 46 mg of a white solid. Data for free base: ¹H NMR (CDCl₃, 400 MHz) δ 7.14 (dd, 1H, J=7.9, 2.1 Hz), 7.07 (d, 1H, J=2.1 Hz), 7.00 (d, 1H, J=7.9 Hz), 2.97-2.91 (m, 4H), 2.83-2.78 (m, 2H), 2.13 (br s, 1H), 2.05-2.00 (m, 2H), 1.78 (br d, 2H, J=10.8 Hz); GCMS m/z 207 (M+).

### Example 8

### 4-Bromo-10-azatricyclo[6.3.2.0^{2,7}]trideca-2,4,6-triene Hydrochloride

A) 1-(4-Bromo-10-aza-tricyclo[6.3.2.0^{2,7}]trideca-2(7),3,5-trien-10-yl)-2,2,2-trifluoro-ethanone

1-(4-Amino-10-aza-tricyclo[6.3.2.0^{2,7}]trideca-2(7),3,5-trien-10-yl)-2,2,2-trifluoro-ethanone (223 mg, 0.785 mmol) was dissolved in H₂O (1.6 mL) and HBr (48% in H₂O, 1.6 mL) and treated with a solution of sodium nitrite (NaNO₂) (108 mg, 1.57 mmol) in H₂O (0.5 mL) dropwise. The resulting yellow solution was added to a solution of CuBr (236 mg, 1.65 mmol) in 48% HBr (aq) solution (1.0 mL) at 0° C. The resulting solution was warmed to 70° C. for 90 minutes (gas evolution), then was cooled to room temperature, diluted with water (20 mL) and extracted with CHCl₃ (4×30 mL). The combined organic extracts were washed with sat. NaHCO₃ then brine and dried (Na₂SO₄), filtered and concentrated. The residue was purified by chromatography (eluting with 10% EtOAc/hexanes) to afford 68 mg of the title compound as an oil: ¹H NMR (CDCl₃, 400 MHz) δ 7.35-7.26 (m, 2H), 7.03-6.98 (m, 1H), 4.19-4.14 (m, 1H), 3.88-3.82 (m, 1H), 3.51-3.46 (m, 1H), 3.40-3.33 (m, 1H), 3.20-3.14 (m, 2H), 2.07-1.97 (m, 2H), 1.79-1.72 (m, 2H); GCMS m/z 347/349 (M+).

B) 4-Bromo-10-azatricyclo[6.3.2.0^{2,7}]dodeca-2(7),3,5-triene hydrochloride

The title compound was prepared from 1-(4-bromo-10-aza-tricyclo[6.3.2.0^{2,7}]trideca-2(7),3,5-trien-10-yl)-2,2,2-trifluoro-ethanone (60 mg, 0.17 mmol) by the method as described in Example 2C to afford 42 mg of a white solid. Data for free base: ¹H NMR (CDCl₃, 400 MHz) δ 7.29 (dd, 1H, J=7.9, 2.1 Hz), 7.22 (d, 1H, J=2.1 Hz), 6.95 (d, 1H, J=7.9 Hz), 2.98-2.90 (m, 4H), 2.82-2.77 (m, 2H), 2.04-2.00 (m, 2H), 1.81-1.77 (m, 2H), 1.65 (br s, 1H); GCMS m/z 251/253 (M+).

### Example 9

### 10-Aza-tricyclo[6.3.2.0^{2,7}]trideca-2,4,6-triene-4-carbonitrile

A) 1-(4-Iodo-10-aza-tricyclo[6.3.2.0^{2,7}]trideca-2(7),3,5-trien-10-yl)-2,2,2-trifluoro-ethanone

1-(4-Amino-10-aza-tricyclo[6.3.2.0^{2,7}]trideca-2(7),3,5-trien-10-yl)-2,2,2-trifluoro-ethanone (475 mg, 1.75 mmol) was dissolved in H₂O (5 mL) and concentrated H₂SO₄ solution (0.5 mL) then cooled to 0° C. and treated with a solution of sodium nitrite (NaNO₂) (133 mg, 1.93 mmol) in H₂O (2 mL) dropwise. Potassium iodide (434 mg, 2.62 mmol) in 1N H₂SO₄ solution (0.5 mL) was added over 10 minutes (reaction becomes dark red). The resulting solution was warmed to room temperature and stirred 18 hours. The reaction was quenched with NaHSO₃ and water (pH 2.5) then extracted with ethyl acetate (4×30 mL). After drying (Na₂SO₄), the solution was filtered and concentrated to a yellow oil which was used without additional purification: GCMS m/z 395 (M+).

B) 4-Iodo-10-aza-tricyclo[6.3.2.0^{2,7}]trideca-2(7),3,5-triene-10-carboxylic acid tert-butyl ester

1-(4-Iodo-10-aza-tricyclo[6.3.2.0^{2,7}]trideca-2(7),3,5-trien-10-yl)-2,2,2-trifluoro-ethanone (540 mg, 1.37 mmol) and 37% saturated aqueous NH₄OH solution (5 mL) were stirred in methanol (25 ml) for 2 hours then concentrated and azeotroped with methanol (2×5 mL). The resulting product was stirred in 1,4-dioxane (20 mL) and treated with saturated Na₂CO₃ solution (20 mL). To this was added di-t-butyldicarbonate (594 mg, 2.72 mmol). After stirring 18 hours the reaction was treated with H₂O (50 mL) and extracted with CH₂Cl₂ (4×30 mL), dried (Na₂SO₄), filtered, concentrated. The resulting oil was used without additional purification: GCMS m/z 399 (M+).

C) 4-Cyano-10-aza-tricyclo[6.3.2.0^{2,7}]trideca-2(7),3,5-triene-10-carboxylic acid tert-butyl ester (Utilizing the methods described in: House, H. O.; Fischer, W. F. J. Org. Chem. 1969, 3626.)

CuCN (242 mg, 2.71 mmol) and KCN (176 mg, 2.71 mmol) were added to a solution of 4-iodo-10-aza-tricyclo[6.3.2.0^{2,7}]trideca-2(7),3,5-triene-10-carboxylic acid tert-butyl ester (542 mg, 1.35 mmol) in DMF (10 mL) and the mixture was stirred for 18 hours at 150° C. The reaction was cooled and diluted with 50% saturated aqueous NaCl solution (25 mL) and extracted with 50% ethyl acetate/hexanes (2×50 mL). After drying (Na₂SO₄), filtration and concentration the product was purified by chromatography (eluting with 25% EtOAc/Hexanes) to give 139 mg of the title compound as an oil: GCMS m/z 298 (M+).

D) 10-Azatricyclo[6.3.2.0^{2,7}]trideca-2,4,6-trien-4-carbonitrile hydrochloride

4-Cyano-10-aza-tricyclo[6.3.2.0^{2,7}]trideca-2(7),3,5-triene-10-carboxylic acid tert-butyl ester (130 mg, 0.436) was treated with 3N HCl ethyl acetate (5 mL) and warmed to reflux for 2 hours, then concentrated, dissolved in a minimum of methanol which was saturated with Et₂O and stirred 18 hours. The product was collected by filtration (80 mg). ¹H NMR (CDCl₃, 400 MHz) δ 7.47 (d, 1H, J=7.5 Hz), 7.39 (s, 1H), 7.21 (d, 1H, J=7.9 Hz), 3.34-3.20 (m, 4H), 3.02 (ap d, 2H, J=12.5 Hz), 2.20 (ap d, 2H, J=9.5 Hz), 1.78 (ap d, 2H, J=10.7 Hz); ¹³C NMR (CDCl₃, 100 MHz) δ 145.3, 141.2, 132.2, 130.3, 127.9, 118.5, 111.7, 48.9, 48.7, 36.4, 35.9, 24.0, 23.9; GCMS m/z 198 (M+).

### Example 10

### 1-(10-Azatricyclo[6.3.2.0^{2,7}]trideca-2(7),3,5-trien-4-yl)-1-ethanone Hydrochloride

A) 1-(4-Acetyl-10-aza-tricyclo[6.3.2.0^{2,7}]trideca-2(7),3,5-trien-10-yl)-2,2,2-trifluoro-ethanone

1-(10-Aza-tricyclo[6.3.2.0^{2,7}]trideca-2(7),3,5-trien-10-yl)-2,2,2-trifluoro-ethanone (1.0 g, 3.71 mmol) and AcCl (2.65 mL, 37.1 mmol) were dissolved in CH₂Cl₂ (20 mL) and treated with aluminum chloride (AlCl₃) (2.47 g, 18.5 mmol). The resulting yellow mixture was stirred for 60 minutes then poured over ice and saturated aqueous NaHCO₃ solution. After stirring 20 minutes the mixture was extracted with CH₂Cl₂ (3×30 mL). The organic layer was dried (Na₂SO₄), filtered and concentrated to a pale yellow oil: APCI MS m/z 312.3 (M+1).

B) 1-(10-Azatricyclo[6.3.2.0^{2,7}]trideca-2(7),3,5-trien-4-yl)-1-ethanone hydrochloride

The title compound was prepared from 1-(4-acetyl-10-aza-tricyclo[6.3.2.0^{2,7}]trideca-2(7),3,5-trien-10-yl)-2,2,2-trifluoro-ethanone (1.15 mg, 3.71 mmol) by the method as described in Example 2C to afford 193 mg of a white solid. Data for free base: ¹H NMR (CD₃OD, 400 MHz) δ 7.95 (dd, 1H, J=7.9, 1.7 Hz), 7.89 (d, 1H, J=1.2 Hz), 7.40 (d, 1H, J=7.9 Hz), 3.45-3.28 (m, 6H), 2.61 (s, 3H), 2.23 (d, 2H, J=9.1 Hz), 1.91-1.88 (m, 2H); ¹³C NMR (CD₃OD, 100 MHz) δ 199.0, 144.8, 139.8, 137.2, 128.4, 127.4, 126.9, 49.6, 49.4, 36.4, 25.6, 25.0, 24.9; GCMS m/z 215 (M+).

### Example 11

### 4,5-Dinitro-10-aza-tricyclo[6.3.2.0^{2,7}]trideca-2(7),3,5-triene

A) 1-(4,5-Dinitro-10-aza-tricyclo[6.3.2.0^{2,7}]trideca-2(7),3,5-trien-10-yl)-2,2,2-trifluoro-ethanone

(Based on the method described in Coon, C. L.; Blucher, W. G.; Hill, M. E. J. Org. Chem., 25, 4243 (1973). For an additional related example of dinitration, see: Tanida, H.; Ishitobi, H.; Irie, T.; Tsushima, T. J. Am. Chem. Soc. 91, 4512 (1969).)

Nitric acid (0.392 ml, 8.35 mmol) was slowly added to a solution of trifluoromethanesulfonic acid (1.48 ml, 16.7 mmol) in CH₂Cl₂ (10.4 ml) at 0° C. with stirring, generating a white precipitate. After 10 minutes, 1-(10-aza-tricyclo[6.3.2.0^{2,7}]trideca-2(7),3,5-trien-10-yl)-2,2,2-trifluoro-ethanone (977 mg, 3.63 mmol) in CH₂Cl₂ (5.6 ml) was added dropwise from an addition funnel over 30 minutes. The reaction was allowed to warm to RT and stirred for 4 h at which time additional nitric acid (0.392 mL) and trifluoromethanesulfonic acid (1.48 mL) were added. The mixture was stirred at RT for 18 h and then poured into a vigorously stirred mixture of H₂O (10 ml) and ice (40 g). The layers were separated and the aqueous layer back extracted with CH₂Cl₂ (3×25 ml). The organic layer was combined and washed with H₂O (3×10 ml). The combined aqueous layers were re-extracted with CH₂Cl₂ (2×25 ml). The organic layer was combined and washed with saturated aqueous NaHCO₃ solution (100 mL) and H₂O (25 mL) dried (Na₂SO₄), filtered and concentrated to solids. Purification of the crude residue by chromatography (elution with 20% EtOAc/Hexanes) afforded 850 mg of the title compound as an orange oil: ¹H NMR (CDCl₃, 400 MHz) δ 7.73 (s, 1H), 7.70 (s, 1H), 4.33-4.28 (m, 1H), 3.97 (dd, 1H, J=14.5, 5.0 Hz)), 3.55 (dd, 1H, J=14.5, 2.1 Hz), 3.44-3.37 (m, 3H), 2.18-2.10 (m, 2H), 1.87-1.77 (m, 2H); GCMS m/z 359 (M⁺).

B) 4,5-Dinitro-10-aza-tricyclo[6.3.2.0^{2,7}]trideca-2(7),3,5-triene

The title compound was prepared from 1-(4,5-Dinitro-10-aza-tricyclo[6.3.2.0^{2,7}]trideca-2(7),3,5-trien-10-yl)-2,2,2-trifluoro-ethanone (110 mg, 0.31 mmol) by the method described in Example 2C to afford 54 mg of a white solid. Data for free base: ¹H NMR (CDCl₃, 400 MHz) δ 7.62 (s, 2H), 3.45 (s, 2H), 3.01-2.91 (m, 4H), 2.22-2.17 (m, 2H), 1.84-1.80 (m, 2H), 1.65 (br s, 1H); ¹³C NMR (CDCl₃, 100 MHz) δ 150.1, 141.5, 122.9, 51.8, 41.7, 25.5; GCMS m/z 263 (M+).

### Example 12

### 5,8,14-Triazatetracyclo[10.3.2.0^{2,11}.0^{4,9}]-heptadeca-2(11),3,5,7,9-pentaene Hydrochloride

A) 1-(4,5-Diamino-10-aza-tricyclo[6.3.2.0^{2,7}]trideca-2(7),3,5-trien-10-yl)-2,2,2-trifluoro-ethanone

Hydrogenation of 1-(4,5-dinitro-10-aza-tricyclo[6.3.2.0^{2,7}]trideca-2(7),3,5-trien-10-yl)-2,2,2-trifluoro-ethanone (1.33 g, 3.70 mmol) under a H₂ atmosphere (50 psi) and 10% Pd/C (5.0 g) in ethanol (100 mL) over 15 hours, followed by filtration through Celite™ and concentration affords 1.1 g of the title compound as a yellow oil: GCMS m/z 299 (M+).

B) 1-(5,8,14-Triazatetracyclo[10.3.2.0^{2,11}.0^{4,9}]heptadeca-2(11),3,5,7,9-pentaene)-2,2,2-trifluoro-ethanone

1-(4,5-Diamino-10-aza-tricyclo[6.3.2.0^{2,7}]trideca-2(7),3,5-trien-10-yl)-2,2,2-trifluoro-ethanone (83.7 mg, 0.28 mmol) was stirred in THF (2.4 ml). This mixture was treated with H₂O (2.4 mL) and glyoxal sodium bisulfite addition compound hydrate (149 mg, 0.56 mmol) then stirred at reflux for 3 hours. The reaction was cooled to room temperature and extracted with ethyl acetate (3×10 ml). The combined organic layer was washed with H₂O (2×10 ml), dried (Na₂SO₄), filtered, concentrated. The residue was purified by chromatography (elution with 1:1 EtOAc/Hexanes) to afford the title compound as a white powder (61 mg): ¹H NMR (CDCl₃, 400 MHz) δ 8.78 (dd, 2H, J=3.7, 2.0 Hz), 7.83 (d, 2H, J=7.9 Hz), 4.37 (ddd, 1H, J=13.7, 5.4, 1.2 Hz), 4.04-4.00 (m, 1H), 3.59-3.39 (m, 4H), 2.15-2.07 (m, 2H), 1.86-1.83 (m, 2H); GCMS m/z 321 (M+).

C) 5,8,14-Triazatetracyclo[10.3.2.0^{2,11}.0^{4,9}]heptadeca-2(11),3,5,7,9-pentaene hydrochloride

The title compound was prepared from 1-(5,8,14-triaza-tetracyclo[10.3.2.0^{2,11}.0^{4,9}]heptadeca-2(11),3,5,7,9-pentaene)-2,2,2-trifluoro-ethanone (61 mg, 0.19 mmol) by the method described in Example 2C to afford 32 mg of a white solid. Data for free base: ¹H NMR (CDCl₃, 400 MHz) δ 8.74 (s, 2H), 7.75 (s, 2H), 3.23 (br s, 2H), 3.08-2.98 (m, 4H), 2.17-2.13 (m, 2H), 1.97 (s, 1H), 1.90-1.84 (m, 2H); ¹³C (CDCl₃ 100 MHz) δ 146.6, 144.3, 142.9,125.7, 53.5, 42.0, 26.0; GCMS m/z 225 (M+).

### Example 13

### 5,7,13-Triazatetracyclo[9.3.2.0^{2,10}.0^{4,8}]-hexadeca-2(10),3,5,8-tetraene Hydrochloride

A) 1-(5,7,13-Triazatetracyclo[9.3.2.0^{2,10}.0^{4,8}]hexadeca-2(10),3,5,8-tetraene)-2,2,2-trifluoro-ethanone

(For conditions, see; Segelstein, B. E.; Chenard, B. L.; Macor, J. E.; Post, R. J. Tetrahedron Lett., 34, 1897 (1993).)

1-(4,5-Diamino-10-aza-tricyclo[6.3.2.0^{2,7}]trideca-2(7),3,5-trien-10-yl)-2,2,2-trifluoro-ethanone (850 mg, 2.84 mmol) was dissolved in ethanol (10 mL) and HOAc (1 mL) and treated with ethoxymethylenemalononitrile (416 mg, 3.41 mmol). The resulting mixture was heated at reflux for 4 hours. The reaction was cooled, concentrated treated with H₂O and saturated aqueous Na₂CO₃ solution and extracted with ethyl acetate (3×50 mL), then dried (Na₂SO₄). After filtration and concentration, the residue was purified by chromatography (elution with EtOAc then 5% MeOH/EtOAc) to afford 877 mg of the title compound: GCMS m/z 309 (M+).

B) 5,7,13-Triazatetracyclo[9.3.2.0^{2,10}.0^{4,8}]hexadeca-2(10),3,5,8-tetraene hydrochloride

The title compound was prepared from 1-(5,7,13-triazatetra-cyclo[9.3.2.0^{2,10}.0^{4,8}]hexadeca-2(10),3,5,8-tetraene)-2,2,2-trifluoro-ethanone (877 mg, 2.83 mmol) by the method described in Example 2C to afford 602 mg of a white solid. Data for free base: ¹H NMR (CD₃OD, 400 MHz) δ 9.41 (s, 1H), 7.81 (s, 2H), 3.65-3.60 (m, 2H), 3.46-3.35 (m, 4H), 2.31-2.27 (m, 2H), 1.94 (br d, 2H, J=9.5 Hz); APCI m/z 214 (M+1).

### Example 14

### 7-Methyl-5,7,13-triazatetracyclo[9.3.2.0^{2,10}.0^{4,8}]-hexadeca-2(10),3,5,8-tetraene Hydrochloride

A) 5,7,13-Triazatetracyclo[9.3.2.0^{2,10}.0^{4,8}]hexadeca-2(10),3,5,8-tetraene-13-carboxylic acid tert-butyl ester

Di-t-butyldicarbonate (616 mg, 2.82 mmol) was added to a solution of 5,7,13-triazatetracyclo[9.3.2.0^{2,10}.0^{4,8}]hexadeca-2(10),3,5,8-tetraene (602 g, 2.82 mmol) in 1,4-dioxane (8 mL), water (2 mL) and 1N NaOH (2 mL). After stirring 18 hours the reaction was treated with sat NaHCO₃ (10 mL) and extracted with CH₂Cl₂ (3×20 mL). The combined organic phases were dried (Na₂SO₄), filtered, concentrated. The crude residue was purified by chromatography to provide the title compound (601 mg) as a yellow waxy solid: ¹H NMR (CDCl₃, 400 MHz) δ 8.09 (s, 1H), 7.44 (s, 1H), 7.31 (s, 1H), 6.04 (br s, 1H), 3.92-3.81 (m, 2H), 3.42-3.38 (m, 2H), 3.20 (s, 2H), 2.06-2.03 (m, 2H), 1.80-1.73 (m, 2H), 1.30 (s, 9H); APCI MS m/z 314.3 (M+1).

B) 7-Methyl-5,7,13-triazatetracyclo[9.3.2.0^{2,10}.0^{4,8}]hexadeca-2(10),3,5,8-tetraene-13-carboxylic acid tert-butyl ester

Iodomethane (17 μL, 0.271 mmol) was added to a solution containing 5,7,13-triazatetracyclo[9.3.2.0^{2,10}.0^{4,8}]hexadeca-2(10),3,5,8-tetraene-13-carboxylic acid tert-butyl ester (85 mg, 0.271 mmol), tetrabutylammonium iodide (1.7 mg, 0.007 mmol), 40% NaOH (aq, 2 mL) and CH₂Cl₂ (2 mL) at RT. The resulting mixture was stirred at RT for 18 hours and diluted with CH₂Cl₂ (10 mL) and water (10 mL). The layers were partitioned and the aqueous layer was extracted with CH₂Cl₂ (2×10 mL). The combined organics were washed with brine, dried (Na₂SO₄), filtered and concentrated. The crude residue was purified by chromatography (elution with EtOAc then 10% MeOH/EtOAc) to afford the title compound (50 mg) as a yellow oil: APCI MS m/z 328.3 (M+1).

C) 7-Methyl-5,7,13-triazatetracyclo[9.3.2.0^{2,10}.0^{4,8}]hexadeca-2(10),3,5,8-tetraene hydrochloride

4N HCl in dioxane (0.5 mL) was added to a solution of 7-methyl-5,7,13-triazatetracyclo[9.3.2.0^{2,10}.0^{4,8}]hexadeca-2(10),3,5,8-tetraene-13-carboxylic acid tert-butyl ester (50 mg, 0.15 mmol) in CH₂Cl₂ (10 mL) and acetone (5 mL) at RT. The mixture was stirred at RT for 18 hr and concentrated to a yellow oil. The oil was stirred in acetone (5 mL) to give a yellow solid which was collected by filtration to give the title compound (15 mg): ¹H NMR (CD₃OD, 400 MHz) δ 9.42 (s, 1H), 7.91 (s, 1H), 7.81 (s, 1H), 4.18 (s, 3H), 3.68-3.65 (m, 2H), 3.48-3.41 (m, 2H), 3.3-3.24 (m, 2H), 2.30 (br d, 2H, J=9.1 Hz), 1.96-1.93 (m, 2H); APCI MS m/z 228.3 (M+1).

### Example 15

### 7-Ethyl-5,7,13-triazatetracyclo[9.3.2.0^{2,10}.0^{4,8}]-hexadeca-2(10),3,5,8-tetraene Hydrochloride

A) 7-Ethyl-5,7,13-triazatetracyclo[9.3.2.0^{2,10}.0^{4,8}]hexadeca-2(10),3,5,8-tetraene-13-carboxylic acid tert-butyl ester

Iodoethane (22 μL, 0.271 mmol) was added to a solution containing 5,7,13-triazatetracyclo[9.3.2.0^{2,10}.0^{4,8}]hexadeca-2(10),3,5,8-tetraene-13-carboxylic acid tert-butyl ester (85 mg, 0.271 mmol), tetrabutylammonium iodide (1.7 mg, 0.007 mmol), 40% NaOH (aq, 2 mL) and CH₂Cl₂ (2 mL) at RT. The resulting mixture was stirred at RT for 18 hours and diluted with CH₂Cl₂ (10 mL) and water (10 mL). The layers were partitioned and the aqueous layer was extracted with CH₂Cl₂ (2×10 mL). The combined organics were washed with brine, dried (Na₂SO₄), filtered and concentrated. The crude residue was purified by chromatography (elution with EtOAc then 10% MeOH/EtOAc) to afford the title compound (72 mg) as a yellow oil: APCI MS m/z 342.3 (M+1).

B) 7-Ethyl-5,7,13-triazatetracyclo[9.3.2.0^{2,10}.0^{4,8}]hexadeca-2(10),3,5,8-tetraene hydrochloride

4N HCl in dioxane (0.5 mL) was added to a solution of 7-ethyl-5,7,13-triazatetracyclo[9.3.2.0^{2,10}.0^{4,8}]hexadeca-2(10),3,5,8-tetraene-13-carboxylic acid tert-butyl ester (72 mg, 0.21 mmol) in CH₂Cl₂ (10 mL) at RT. The mixture was stirred at RT for 18 hr and concentrated to a yellow oil to give the title compound (25 mg): ¹³C NMR (CD₃OD, 100 MHz) δ 143.3, 139.6, 139.1, 131.5, 131.2, 115.5, 115.4, 53.9, 53.2, 47.1, 37.9, 37.3, 26.7, 26.6, 17.3; APCI MS m/z 242.3 (M+1).

### Example 16

### 7-Propyl-5,7,13-triazatetracyclo[9.3.2.0^{2,10}.0^{4,8}]-hexadeca-2(10),3,5,8-tetraene Hydrochloride

A) 7-Propyl-5,7,13-triazatetracyclo[9.3.2.0^{2,10}.0^{4,8}]hexadeca-2(10),3,5,8-tetraene-13-carboxylic acid tert-butyl ester

Iodopropane (26 μL, 0.271 mmol) was added to a solution containing 5,7,13-triazatetracyclo[9.3.2.0^{2,10}.0^{4,8}]hexadeca-2(10),3,5,8-tetraene-13-carboxylic acid tert-butyl ester (85 mg, 0.271 mmol), tetrabutylammonium iodide (1.7 mg, 0.007 mmol), 40% NaOH (aq, 2 mL) and CH₂Cl₂ (2 mL) at RT. The resulting mixture was stirred at RT for 18 hours and diluted with CH₂Cl₂ (10 mL) and water (10 mL). The layers were partitioned and the aqueous layer was extracted with CH₂Cl₂ (2×10 mL). The combined organics were washed with brine, dried (Na₂SO₄), filtered and concentrated. The crude residue was purified by chromatography (elution with EtOAc then 10% MeOH/EtOAc) to afford the title compound (33 mg) as a yellow oil: APCI MS m/z 356.4 (M+1).

B) 7-Propyl-5,7,13-triazatetracyclo[9.3.2.0^{2,10}.0^{4,8}]hexadeca-2(10),3,5,8-tetraene hydrochloride

4N HCl in dioxane (0.5 mL) was added to a solution of 7-ethyl-5,7,13-triazatetracyclo[9.3.2.0^{2,10}.0^{4,8}]hexadeca-2(10),3,5,8-tetraene-13-carboxylic acid tert-butyl ester (33 mg, 0.21 mmol) in CH₂Cl₂ (10 mL) at RT. The mixture was stirred at RT for 18 hr and concentrated to a yellow oil to give the title compound (27 mg): ¹H NMR (CD₃OD, 400 MHz) δ 9.50 (br s, 1H), 7.94 (br s, 1H), 7.80 (br s, 1H), 4.52 (br s, 2H), 3.63 (br s, 2H), 3.50-3.30 (m, 4H), 2.37-2.25 (m, 2H), 2.06-1.94 (m, 4H), 1.03 (t, 3H, J=6.2 Hz); APCI MS m/z 256.4 (M+1).

### Example 17

### 6-Methyl-5,7,13-triazatetracyclo[9.3.2.0^{2,10}.0^{4,8}]-hexadeca-2(10),3,5,8-tetraene Hydrochloride

A) 1-(6-Methyl-5,7,13-triazatetracyclo[9.3.2.0^{2,10}.0^{4,8}]hexadeca-2(10),3,5,8-tetraene)-2,2,2-trifluoro-ethanone

(For conditions, see; Segelstein, B. E.; Chenard, B. L.; Macor, J. E.; Post, R. J. Tetrahedron Lett., 34, 1897 (1993).)

1-(4,5-Diamino-10-aza-tricyclo[6.3.2.0^{2,7}]trideca-2(7),3,5-trien-10-yl)-2,2,2-trifluoro-ethanone (417 mg, 1.39 mmol) was dissolved in ethanol (10 mL) and HOAc (1 mL) and treated with 1-ethoxyethylidene malononitrile (227 mg, 1.67 mmol). The resulting mixture was heated at reflux for 4 hours. The reaction was cooled, concentrated treated with H₂O and saturated aqueous Na₂CO₃ solution and extracted with ethyl acetate (3×50 mL), then dried (Na₂SO₄). After filtration and concentration, the residue was purified by chromatography (elution with EtOAc then 5% MeOH/EtOAc) to afford the title compound (448 mg) as an oil: GCMS m/z 323 (M+).

B) 6-Methyl-5,7,13-triazatetracyclo[9.3.2.0^{2,10}.0^{4,8}]hexadeca-2(10),3,5,8-tetraene hydrochloride

The title compound was prepared from 1-(6-methyl-5,7,13-triazatetracyclo[9.3.2.0^{2,10}.0^{4,8}]hexadeca-2(10),3,5,8-tetraene)-2,2,2-trifluoro-ethanone (877 mg, 2.83 mmol) by the method described in Example 2C to afford 313 mg of a white solid. Data for free base: ¹H NMR (CD₃OD, 400 MHz) δ 7.68 (s, 2H), 3.59 (br s, 2H), 3.44-3.34.(m 4H), 2.87 (s, 3H), 2.27 (d, 2H, J=9.1 Hz), 1.94 (br d, 2H, J=9.9 Hz); ¹³C NMR (CD₃OD, 100 MHz) δ 151.2, 137.9, 131.8, 112.1, 49.9, 36.7, 25.1, 11.5; APCI m/z 228.2 (M+1).

### Example 18

### 6,7-Dimethyl-5,7,13-triazatetracyclo[9.3.2.0^{2,10}.0^{4,8}]-hexadeca-2(10),3,5,8-tetraene Hydrochloride

A) 6-Methyl-5,7,13-triazatetracyclo[9.3.2.0^{2,10}.0^{4,8}]hexadeca-2(10),3,5,8-tetraene-13-carboxylic acid tert-butyl ester

Di-t-butyldicarbonate (331 mg, 1.51 mmol) was added to a solution of 6-methyl-5,7,13-triazatetracyclo[9.3.2.0^{2,10}.0^{4,8}]hexadeca-2(10),3,5,8-tetraene (313 g, 1.38 mmol) in 1,4-dioxane (8 mL), water (2 mL) and 1N NaOH (2 mL). After stirring 18 hours the reaction was treated with sat NaHCO₃ (10 mL) and extracted with CH₂Cl₂ (3×20 mL). The combined organic phases were dried (Na₂SO₄), filtered, concentrated. The crude residue was purified by chromatography to provide the title compound (257 mg) as a yellow waxy solid: APCI MS m/z 328.3 (M+1).

B) 6,7-Dimethyl-5,7,13-triazatetracyclo[9.3.2.0^{2,10}.0^{4,8}]hexadeca-2(10),3,5,8-tetraene-13-carboxylic acid tert-butyl ester

Iodomethane (20 μL, 0.336 mmol) was added to a solution containing 6-methyl-5,7,13-triazatetracyclo[9.3.2.0^{2,10}.0^{4,8}]hexadeca-2(10),3,5,8-tetraene-13-carboxylic acid tert-butyl ester (85 mg, 0.271 mmol), tetrabutylammonium iodide (1.6 mg, 0.006 mmol), 40% NaOH (aq, 1 mL) and toluene (1 mL) at RT. The resulting mixture was stirred at RT for 18 hours and diluted with CH₂Cl₂ (10 mL) and water (10 mL). The layers were partitioned and the aqueous layer was extracted with CH₂Cl₂ (2×10 mL). The combined organics were washed with brine, dried (Na₂SO₄), filtered and concentrated. The crude residue was purified by chromatography (elution with EtOAc then 10% MeOH/EtOAc) to afford the title compound (24 mg) as a yellow oil: APCI MS m/z 342.3 (M+1).

C) 6,7-Dimethyl-5,7,13-triazatetracyclo[9.3.2.0^{2,10}.0^{4,8}]hexadeca-2(10),3,5,8-tetraene hydrochloride

4N HCl in dioxane (0.5 mL) was added to a solution of 6,7-dimethyl-5,7,13-triazatetracyclo[9.3.2.0^{2,10}.0^{4,8}]hexadeca-2(10),3,5,8-tetraene-13-carboxylic acid tert-butyl ester (24 mg, 0.07 mmol) in CH₂Cl₂ (5 mL) at RT. The mixture was stirred at RT for 18 hr and concentrated to a yellow oil to give the title compound (15 mg): ¹H NMR (CD₃OD, 400 MHz) δ 7.78 (br s, 1H), 7.67 (br s, 1H), 4.00 (s, 3H), 3.63-3.58 (m, 2H), 3.40-3.32 (m, 4H), 2.87 (s, 3H), 2.27 (br d, 2H, J=8.7 Hz), 1.92 (br d, 2H, J=8.7 Hz); APCI MS m/z 242.3 (M+1).

### Example 19

### 6-Methyl-7-ethyl-5,7,13-triazatetracyclo[9.3.2.0^{2,10}.0^{4,8}]-hexadeca-2(10),3,5,8-tetraene Hydrochloride

A) 6-Methyl-7-ethyl-5,7,13-triazatetracyclo[9.3.2.0^{2,10}.0^{4,8}]hexadeca-2(10),3,5,8-tetraene-13-carboxylic acid tert-butyl ester

Iodoethane (20 μL, 0.26 mmol) was added to a solution containing 6-methyl-5,7,13-triazatetracyclo[9.3.2.0^{2,10}.0^{4,8}]hexadeca-2(10),3,5,8-tetraene-13-carboxylic acid tert-butyl ester (85 mg, 0.26 mmol), tetrabutylammonium iodide (1.6 mg, 0.007 mmol), 40% NaOH (aq, 2 mL) and CH₂Cl₂ (2 mL) at RT. The resulting mixture was stirred at RT for 18 hours and diluted with CH₂Cl₂ (10 mL) and water (10 mL). The layers were partitioned and the aqueous layer was extracted with CH₂Cl₂ (2×10 mL). The combined organics were washed with brine, dried (Na₂SO₄), filtered and concentrated. The crude residue was purified by chromatography (elution with EtOAc then 10% MeOH/EtOAc) to afford the title compound (63 mg) as a yellow oil: APCI MS m/z 356.4 (M+1).

B) 6-Methyl-7-ethyl-5,7,13-triazatetracyclo[9.3.2.0^{2,10}.0^{4,8}]hexadeca-2(10),3,5,8-tetraene hydrochloride

4N HCl in dioxane (0.5 mL) was added to a solution of 6-methyl-7-ethyl-5,7,13-triazatetracyclo[9.3.2.0^{2,10}.0^{4,8}]hexadeca-2(10),3,5,8-tetraene-13-carboxylic acid tert-butyl ester (63 mg, 0.18 mmol) in CH₂Cl₂ (10 mL) at RT. The mixture was stirred at RT for 18 hr and concentrated to a yellow oil to give the title compound (22 mg): APCI MS m/z 256.3 (M+1).

### Example 20

### 6-Methyl-7-propyl-5,7,13-triazatetracyclo[9.3.2.0^{2,10}.0^{4,8}]-hexadeca-2(10),3,5,8-tetraene Hydrochloride

A) 6-Methyl-7-propyl-57,13-triazatetracyclo[9.3.2.0^{2,10}.0^{4,8}]hexadeca-2(10),3,5,8-tetraene-13-carboxylic acid tert-butyl ester

Iodopropane (25 μL, 0.26 mmol) was added to a solution containing 6-methyl-5,7,13-triazatetracyclo[9.3.2.0^{2,10}.0^{4,8}]hexadeca-2(10),3,5,8-tetraene-13-carboxylic acid tert-butyl ester (85 mg, 0.26 mmol), tetrabutylammonium iodide (1.6 mg, 0.006 mmol), 40% NaOH (aq, 2 mL) and CH₂Cl₂ (2 mL) at RT. The resulting mixture was stirred at RT for 18 hours and diluted with CH₂Cl₂ (10 mL) and water (10 mL). The layers were partitioned and the aqueous layer was extracted with CH₂Cl₂ (2×10 mL). The combined organics were washed with brine, dried (Na₂SO₄), filtered and concentrated. The crude residue was purified by chromatography (elution with EtOAc then 10% MeOH/EtOAc) to afford the title compound (35 mg) as a yellow oil: APCI MS m/z 370.4 (M+1).

B) 6-Methyl-7-propyl-5,7,13-triazatetracyclo[9.3.2.0^{2,10}.0^{4,8}]hexadeca-2(10),3,5,8-tetraene hydrochloride

4N HCl in dioxane (0.5 mL) was added to a solution of 6-methyl-7-ethyl-5,7,13-triazatetracyclo[9.3.2.0^{2,10}.0^{4,8}]hexadeca-2(10),3,5,8-tetraene-13-carboxylic acid tert-butyl ester (33 mg, 0.09 mmol) in CH₂Cl₂ (10 mL) at RT. The mixture was stirred at RT for 18 hr and concentrated to a yellow oil to give the title compound (28 mg): ¹H NMR (CD₃OD, 400 MHz) δ 7.85 (br s, 1H), 7.70 (br s, 1H), 4.44 (br s, 2H), 3.64-3.59 (m, 2H), 3.45-3.35 (m, 4H), 2.90 (s, 3H), 2.35-2.25 (m, 2H), 2.01-1.92 (m, 4H), 1.05-1.02 (m, 3H); APCI MS m/z 270.4 (M+1).

### Example 21

### 6-Methyl-5-oxa-7,13-diazatetracyclo[9.3.2.0^{2,10}.0^{4,8}]-hexadeca-2(10),3,6,8-tetraene Hydrochloride

A) 2,2,2-Trifluoro-1-(4-hydroxy-5-nitro-10-aza-tricyclo[6.3.2.0^{2,7}]trideca-2(7),3,5-trien-10-yl)-ethanone

1-(4,5-Dinitro-10-aza-tricyclo[6.3.2.0^{2,7}]trideca-2(7),3,5-trien-10-yl)-2,2,2-trifluoro-ethanone (900 mg, 2.50 mmol) and potassium acetate (KOAC) (246 mg, 2.50 mmol) were dissolved in DMSO (10 mL) and warmed with stirring to 100° C. for 16 hours. The mixture was cooled and diluted with H₂O (50 mL) then extracted with 80% ethyl acetate/hexanes (6×25 mL). The organic layer was washed with H₂O (3×20 mL), dried (Na₂SO₄), filtered and concentrated. The crude residue was purified by chromatography (elution with 20% EtOAc/Hexanes to 40% EtOAc/Hexanes) to give the title compound as an oil (150 mg): GCMS m/z 330 (M+).

B) 2,2,2-Trifluoro-1-(4-hydroxy-5-amino-10-aza-tricyclo[6.3.2.0^{2,7}]trideca-2(7),3,5-trien-10-yl)-ethanone

2,2,2-Trifluoro-1-(4-hydroxy-5-nitro-10-aza-tricyclo[6.3.2.0^{2,7}]trideca-2(7),3,5-trien-10-yl)-ethanone (150 mg, 0.45 mmol) was hydrogenated in ethanol (25 mL) under a H₂ atmosphere at (45 psi) over 10% Pd/C (50 mg) for 3.5 hours then filtered through a Celite™ pad and concentrated to afford the title compound as a yellow oil (140 mg): GCMS m/z 300 (M⁺).

C) 2,2,2-Trifluoro-1-(6-methyl-5-oxa-7,13-diazatetracyclo[9.3.2.0^{2,10}.0^{4,8}]hexadeca-2(10),3,6,8-tetraene)-ethanone

2,2,2-Trifluoro-1-(4-hydroxy-5-amino-10-aza-tricyclo[6.3.2.0^{2,7}]trideca-2(7),3,5-trien-10-yl)-ethanone (140 mg, 0.524 mmol), triethyl orthoacetate (0.34 mL, 1.83 mmol), pyridinium-p-toluenesulfonic acid (PPTS, 20 mg, 0.08 mmol) and xylenes (10 mL) were combined under nitrogen and stirred at 135° C. for 18 hours. The mixture was cooled, treated with H₂O and extracted with ethyl acetate. The extracts were dried (Na₂SO₄), filtered, concentrated and purified by chromatography (elution with 10% EtOAc/Hexanes) to give the title compound (40 mg) as an oil: GCMS m/z 324.

D) 6-Methyl-5-oxa-7,13-diazatetracyclo[9.3.2.0^{2,10}.0^{4,8}]hexadeca-2(10),3,6,8-tetraene hydrochloride

The title compound was prepared from 2,2,2-trifluoro-1-(6-methyl 5-oxa-7,13-diazatetracyclo[9.3.2.0^{2,10}.0^{4,8}]hexadeca-2(10),3,6,8-tetraene)-ethanone (40 mg, 0.12 mmol) by the method described in Example 2C to afford 25 mg of a white solid. Data for free base: ¹H NMR (CD₃OD, 400 MHz) δ 7.66-7.64 (m, 2H), 3.61-3.54 (m, 2H), 3.42-3.25 (m, 4H), 2.80 (s, 3H), 2.32-2.25 (m, 2H), 1.95-1.85 (m, 2H); GCMS m/z 228 (M+).

### Example 22

### 12-Exo-methyl-4-nitro-10-aza-tricyclo[6.3.1.0^{2,7}]dodeca-2(7),3,5-triene Hydrochloride

A) 3-Cyano-2-methyl-3-phenyl-propionic acid methyl ester

Benzyl cyanide (0.5 mL, 4.34 mmol) was slowly added to a slurry of NaH (60%, 182 mg, 4.56 mmol) in THF (9 mL) at RT. Methyl 2-bromopropionate (0.48 mL, 4.34 mmol) was added to this mixture and the resulting solution was stirred at RT for 18 h. The mixture was treated with saturated (aq) NaHCO₃ (5 mL) and the resulting mixture was extracted with EtOAc (3×10 mL). The combined organics were washed with brine, dried (Na₂SO₄), filtered and concentrated. The crude residue was purified by chromatography (eluting with 5% EtOAc/hexanes) to afford the title compound (577 mg) as a mixture of diastereomers: ¹³C NMR (CDCl₃, 100 MHz) δ 173.3, 173.2, 134.0, 133.0, 129.3, 128.9, 128.7, 128.4, 128.0, 119.8, 119.1, 52.53, 52.50, 45.0, 44.5, 40.5, 40.1, 15.1, 14.7; GCMS m/z 203 (M+).

B) 3-Cyano-2-methyl-3-phenyl-propionic acid

Lithium iodide (836 mg, 6.25 mmol) was added to a solution of 3-cyano-2-methyl-3-phenyl-propionic acid methyl ester (577 mg, 2.84 mmol) in collidine (14.2 mL). The resulting mixture was heated at reflux for 18 h. The mixture was cooled in an ice/water bath and a solution of H₂SO₄ (7.7 mL) in water (27 mL) was slowly added over 30 min. The resulting mixture was extracted with ether (2×100 mL), and the combined organics were dried (MgSO₄), filtered and concentrated. The residue was dissolved in ether (50 mL) and the resulting solution was washed with 1 N NaOH (3×5 mL). The combined aqueous phases were washed with ether (2×25 mL), acidified with 6 N HCl and extracted with EtOAc (3×50 mL). The combined organics were dried (Na₂SO₄), filtered and concentrated to afford the title compound (484 mg) as a mixture of diastereomers: GCMS m/z 189 (M+).

C) 2-Methyl-3-oxo-indan-1-carboxylic acid methyl ester

3-Cyano-2-methyl-3-phenyl-propionic acid (480 mg, 2.54 mmol) was dissolved in H₂SO₄ (2.54 mL) and heated in an oil bath at 90° C. for 18 h. Cool to RT and slowly pour the resulting mixture into methanol (10 mL) cooled in an ice/water bath. The resulting mixture was heated to reflux for 3 h. Upon cooling to RT the mixture was poured onto ice (50 g). The resulting aqueous solution was extracted with EtOAc (3×30 mL). Wash the combined organic phases with sat. NaHCO₃ (3×10 mL), water (10 mL) then brine (10 mL). The organics were dried (Na₂SO₄), filtered and concentrated to provide the title compound (208 mg) as a 8:1 mixture (trans/cis) of diastereomers as determined by ¹H NMR: ¹H NMR (CDCl₃, 400 MHz) δ 7.74-7.70 (m, 1H), 7.62-7.56 (m, 2H), 7.46-7.37 (m, 1H), 4.36 (d, J=8.3 Hz, minor), 3.81 (d, 1H, J=4.5 Hz, major), 3.76 (s, 3H, major), 3.64 (s, minor), 3.09-3.02 (m, 1H, major), 2.98-2.90 (m, minor), 1.33 (d, 3H, J=7.5 Hz, major), 1.21 (d, J=7.5 Hz, minor); GCMS m/z 204 (M+).

D) 3-Cyano-2-methyl-3-trimethylsilanyloxy-indan-1-carboxylic acid methyl ester

2-Methyl-3-oxo-indan-1-carboxylic acid methyl ester (200 mg, 0.98 mmol) was dissolved in CH₂Cl₂ (1.0 mL). ZnI₂ (1.6 mg, 0.005 mmol), and I₂ (1.5 mg, 0.006 mmol) were added and then TMSCN (0.261 mL, 1.96 mmol) was added dropwise over 15 min. The resulting mixture was heated at reflux for 20 h. The mixture was cooled to RT and sat. NaHCO₃ (10 mL) and CHCl₃ (10 mL) were added, and the resulting mixture was stirred for 30 min. The mixture was partitioned and the organic layer was washed successively with sat. NaHCO₃ (10 mL), water (10 mL) and brine (10 mL). The organic layer was dried (Na₂CO₃), filtered and concentrated to afford 312 mg of the title compound as a brown oil which was used without further purification: GCMS m/z 303 (M+).

E) 12-Exo-methyl-10-aza-tricyclo[6.3.1.0^{2,7}]dodeca-2,4,6-trien-9-one

Pearlman's catalyst (20% Pd(OH)₂—C (50% water), 206 mg, 0.15 mmol) was added to a solution of 3-cyano-2-methyl-3-trimethylsilanyloxy-indan-1-carboxylic acid methyl ester (312 mg, 0.98 mmol) in MeOH (10 mL) and conc. H₂SO₄ (0.1 mL). This mixture was shaken under an atmosphere of hydrogen (50 psi) at 50° C. for a period of 20 h. The resulting solution was filtered through a pad of Celite™ and washed with MeOH (10 mL). Sodium tert-butoxide (282 mg, 2.94 mmol) was added and the resulting solution was stirred at RT for 20 h. The mixture was treated with sat. aq. NH₄Cl (1.0 mL) and partitioned between EtOAc (10 mL) and water (10 mL). The layers were separated and the aqueous layer was extracted with EtOAc (3×10 mL). The combined organics were washed with brine, dried (Na₂SO₄), filtered and concentrated. The crude residue was purified by chromatography (elution with 2% MeOH/CHCl₃ with 0.1% NH₄OH) to afford the title compound (75 mg) as a film: ¹H NMR (CDCl₃, 400 MHz) δ 7.27-7.24 (m, 2H), 7.19-7.11 (m, 2H), 6.55 (br s, 1H), 3.58 (ddd, 1H, J=11.2, 4.2, 1.2 Hz), 3.18 (s, 1H), 3.10-3.06 (m, 1H), 3.00 (d, 1H, J=3.7 Hz), 2.65 (q, 1H, J=6.6 Hz), 0.80 (d, 3H, J=6.6 Hz); ¹³C NMR (CDCl₃, 100 MHz) δ 174.5, 143.04, 143.01, 128.0, 127.6, 124.2, 123.9, 55.6, 47.9, 45.6, 44.0, 16.8; GCMS m/z 187 (M+).

F) 2,2,2-Trifluoro-1-(12-exo-methyl-10-aza-tricyclo[6.3.1.0^{2,7}]dodeca-2,4,6-trien-10-yl)-ethanone

Lithium aluminum hydride (1 M in THF, 0.8 mL, 0.8 mmol) was added to a solution of 12-exo-methyl-10-aza-tricyclo[6.3.1.0^{2,7}]dodeca-2,4,6-trien-9-one (75 mg, 0.40 mmol) in THF (0.8 mL) at 45° C. The resulting mixture was stirred for 9.5 h, cooled to RT and treated with a solution of water (58 μL) in THF (0.3 mL). The resulting slurry was stirred at RT for 15 h, filtered through a pad of Celite™, washing the filter cake with THF (10 mL). The filtrate was concentrated and the resulting residue was dissolved in CH₂Cl₂ (1.3 mL). Pyridine (81 μL, 1.0 mmol) was added to the mixture followed by trifluoroacetic anhydride (71 μL, 0.5 mmol) at RT. This mixture stirred for 20 h and was diluted with CHCl₃ (10 mL) and 1 M HCl (5 mL). The mixture was separated and the aqueous layer was extracted with CHCl₃ (3×5 mL). The combined organic extracts were washed with 1 M HCl (10 mL), water (10 mL) then brine (10 mL). The organics were dried (Na₂SO₄), filtered and concentrated. The crude residue was purified by chromatography (elution with 5% EtOAc/hexanes) to afford the title compound (61 mg) as a white solid: ¹H NMR (CDCl₃, 400 MHz) δ 7.22-7.19 (m, 4H), 4.34 (dd, 1H, J=12.9, 2.1 Hz), 3.92-3.88 (m, 1H), 3.52 (dd, 1H, J=12.4, 1.7 Hz), 3.12 (d, 1H, J=12.9 Hz), 2.97-2.96 (m, 1H), 2.92-2.91 (m, 1H), 2.27 (q, 1H, J=6.6 Hz), 0.90 (d, 3H, J=6.6 Hz); ¹³C NMR (CDCl₃, 100 MHz) δ 157.8, 157.4, 142.4, 141.6,m 128.1, 127.8, 124.3, 124.1, 120.8, 117.9, 115.1, 112.2, 51.0, 48.9, 48.6, 46.7, 46.4, 18.1; GCMS m/z 269 (M+).

G) 2,2,2-Trifluoro-1-(12-exo-methyl-4-nitro-10-aza-tricyclo[6.3.1.0^{2,7}]dodeca-2,4,6-trien-10-yl)-ethanone

Nitric acid (36 μL, 0.40 mmol, 69%) was slowly added to a solution of 2,2,2-trifluoro-1-(12-exo-methyl-10-aza-tricyclo[6.3.1.0^{2,7}]dodeca-2,4,6-trien-10-yl)-ethanone (33 mg, 0.12 mmol) in TFA (0.12 mL) at 0° C. (ice bath). The mixture was allowed to warm to RT and stirred for 4 h at which time it was poured over CHCl₃ (10 mL) and water (10 mL). The solution was neutralized with sat. NaHCO₃ (aq) and partitioned. The aqueous layer was extracted with CHCl₃ (3×10 mL). The combined organics were washed with water (10 mL) then brine (10 mL) and dried (Na₂SO₄), filtered and concentrated to afford the title compound (33 mg) which was used without further purification: ¹H NMR (CDCl₃, 400 MHz) δ 8.12 (d, 1H, J=8.3 Hz), 8.08 (d, 1H, J=4.6 Hz), 7.38 (d, 1H, J=8.3 Hz), 4.40 (dd, 1H, J=12.9, 2.1 Hz), 3.96 (d, 1H, 12.4 Hz), 3.59 (dd, 1H, J=12.9, 1.2 Hz), 3.20 (d, 1H, J=12.9 Hz), 3.13-3.05 (m, 2H), 2.40 (q, 1H, J=6.6 Hz), 0.90 (d, 3H, J=6.6 Hz); GCMS m/z 314 (M+).

H) 12-Exo-methyl-4-nitro-10-aza-tricyclo[6.3.1.0^{2,7}]dodeca-2(7),3,5-triene hydrochloride

The title compound was prepared from 2,2,2-trifluoro-1-(12-exo-methyl-4-nitro-10-aza-tricyclo[6.3.1.0^{2,7}]dodeca-2,4,6-trien-10-yl)-ethanone (30 mg, 0.096 mmol) by the method described in Example 2C to afford 21 mg of a white solid. Data for free base: ¹H NMR (CDCl₃, 400 MHz) δ 8.11 (dd, 1H, J=7.9, 2.1 Hz), 8.03 (d, 1H, J=2.1 Hz), 7.31 (d, 1H, J=8.3 Hz), 3.05-3.01 (m, 2H), 2.85-2.81 (m, 4H), 2.27 (q, 1H, J=6.6 Hz), 1.61 (br s, 1H), 0.84 (d, 3H, J=6.6 Hz); ¹³C NMR (CDCl₃, 100 MHz) δ 152.6, 147.9, 146.3, 124.3, 123.6, 119.1, 50.2, 50.0, 49.9, 49.4, 49.2, 19.0; GCMS m/z 218 (M+).

### Example 23

### 12,12-Dimethyl-10-aza-tricyclo[6.3.1.0^{2,7}]dodeca-2(7),3,5-triene Hydrochloride

A) 3-Cyano-2,2-dimethyl-3-phenyl-propionic acid

Benzyl cyanide (5.0 mL, 43.4 mmol) was slowly added to a slurry of NaH (60%, 1.74 g, 43.4 mmol) in THF (87 mL) at RT. Methyl 2-bromo-2-methylpropionate (5.58 mL, 43.4 mmol) was added to this mixture and the resulting solution was stirred at RT for 18 h. The mixture was treated with saturated (aq) NaHCO₃ (25 mL) and the resulting mixture was extracted with EtOAc (3×100 mL). The combined organics were washed with brine, dried (Na₂SO₄), filtered and concentrated to give 3-cyano-2,2-dimethyl-3-phenyl-propionic acid methyl ester [GCMS m/z 217 (m+)] as a brown oil. This material was dissolved in collidine (220 mL) and lithium iodide (12.8 g, 95.5 mmol) was added. The resulting mixture was heated at reflux for 18 h. The mixture was cooled in an ice/water bath and a solution of H₂SO₄ (120 mL) in water (400 mL) was slowly added over 90 min. The resulting mixture was extracted with ether (5×200 mL), and the combined organics were dried (MgSO₄), filtered and concentrated. The resulting oil was dissolved in ether (100 mL) and the resulting solution was washed with 1 N NaOH (2×50 mL, then 1×25 mL). The combined aqueous phases were washed with ether (2×100 mL), acidified with 6 N HCl (˜30 mL) and extracted with EtOAc (3×100 mL). The combined organics were dried (Na₂SO₄), filtered and concentrated to afford the title compound (6.31 g) as a yellow oily solid: ¹H NMR (CDCl₃, 400 MHz) 11.3 (br s, 1H), 7.38-7.32 (m, 5H), 4.32 (s, 1H), 1.47 (s, 3H), 1.22 (s, 3H); GCMS m/z 203 (M+).

B) 2,2-Dimethyl-3-oxo-indan-1-carboxylic acid methyl ester

3-Cyano-2,2-dimethyl-3-phenyl-propionic acid (6.31 g, 31.1 mmol) was dissolved in H₂SO₄ (31 mL) and heated in an oil bath at 90° C. for 18 h. Cool to RT and slowly pour the resulting mixture into methanol (62 mL) cooled in an ice/water bath. The resulting mixture was heated to reflux for 3 h. Upon cooling to RT the mixture was poured onto ice (250 g). The resulting aqueous solution was extracted with EtOAc (3×100 mL). Wash the combined organic phases with sat. NaHCO₃ (3×50 mL), water (50 mL) then brine (50 mL). The organics were dried (Na₂SO₄), filtered and concentrated to provide the title compound (4.17 g) as a yellow oil: ¹H NMR (CDCl₃, 400 MHz) δ 7.77 (d, 1H, J=7.9 Hz), 7.66-7.62 (m, 1H), 7.50 (dd, 1H, J=7.9, 0.8 Hz), 7.47-7.43 (m, 1H), 3.98 (s, 1H), 3.75 (s, 3H), 1.36 (s, 3H), 1.12 (s, 3H); ¹³C NMR (CDCl₃, 100 MHz) δ 208.5, 172.0, 149.2, 135.3, 128.9, 127.6, 124.6, 56.7, 52.2, 49.6, 25.4, 21.6; GCMS m/z 218 (M+).

C) 3-Cyano-2,2-dimethyl-3-trimethylsilanyloxy-indan-1-carboxylic acid methyl ester

2,2-Dimethyl-3-oxo-indan-1-carboxylic acid methyl ester (1.0 g, 4.6 mmol) was dissolved in CH₂Cl₂ (4.6 mL). ZnI₂ (7.3 mg, 0.023 mmol), and I₂ (7.0 mg, 0.028 mmol) were added and then TMSCN (1.22 mL, 9.17 mmol) was added dropwise over 15 min. The resulting mixture was heated at reflux for 20 h. The mixture was cooled to RT and sat. NaHCO₃ (20 mL) and CHCl₃ (20 mL) was added, and the resulting mixture was stirred for 30 min. The mixture was partitioned and the organic layer was washed successively with sat. NaHCO₃ (20 mL), water (20 mL) and brine (20 mL). The organic layer was dried (Na₂CO₃), filtered and concentrated to afford 1.45 g of the title compound as a brown oil which was used without further purification: GCMS m/z 317 (M+).

D) 12,12-dimethyl-10-aza-tricyclo[6.3.1.0^{2,7}]dodeca-2,4,6-trien-9-one

Pearlman's catalyst (20% Pd(OH)₂—C (50% water), 825 mg, 0.59 mmol) was added to a solution of 3-cyano-2,2-dimethyl-3-trimethylsilanyloxy-indan-1-carboxylic acid methyl ester (1.25 g, 3.93 mmol) in MeOH (10 mL) and conc. H₂SO₄ (0.3 mL). This mixture was shaken under an atmosphere of hydrogen (50 psi) at 50° C. for a period of 20 h. The resulting solution was filtered through a pad of Celite™ and washed with MeOH (10 mL). Sodium tert-butoxide (1.13 g, 11.7 mmol) was added and the resulting solution was stirred at reflux for 20 h. The mixture was treated with sat. aq. NH₄Cl (5 mL) and partitioned between EtOAc (10 mL) and water (10 mL). The layers were separated and the aqueous layer was extracted with EtOAc (3×10 mL). The combined organics were washed with brine, dried (Na₂SO₄), filtered and concentrated. The crude residue was purified by chromatography (elution with 2% MeOH/CHCl₃ with 0.1% NH₄OH) to afford the title compound (120 mg) as a film: ¹H NMR (CDCl₃, 400 MHz) δ 7.31 (d, 1H, J=6.6 Hz), 7.24-7.13 (m, 3H), 3.62 (dd, 1H, J=12.2, 4.2 Hz), 3.07 (dd, 1H, J=12.2, 1.4 Hz), 3.04 (s, 1H), 2.82 (d, 1H, J=4.2 Hz), 1.30 (s, 3H), 0.92 (s, 3H); GCMS m/z 201 (M+).

E) 2,2,2-Trifluoro-1-(12,12-dimethyl-10-aza-tricyclo[6.3.1.0^{2,7}]dodeca-2,4,6-trien-10-yl)-ethanone

Lithium aluminum hydride (1 M in THF, 1.4 mL, 1.4 mmol) was added to a solution of 12,12-dimethyl-10-aza-tricyclo[6.3.1.0^{2,7}]dodeca-2,4,6-trien-9-one (115 mg, 0.57 mmol) in THF (1.2 mL) at 45° C. The resulting mixture was stirred for 18 h, cooled to RT and treated with a solution of water (100 μL) in THF (0.92 mL). The resulting slurry was stirred at RT for 30 min, filtered through a pad of Celite™, washing the filter cake with THF (10 mL). The filtrate was concentrated and the resulting residue was dissolved in CH₂Cl₂ (1.9 mL). Pyridine (116 μL, 1.43 mmol) was added to the mixture followed by trifluoroacetic anhydride (100 μL, 0.72 mmol) at RT. This mixture stirred for 1 h and was diluted with CHCl₃ (10 mL) and 1 M HCl (5 mL). The mixture was separated and the aqueous layer was extracted with CHCl₃ (3×5 mL). The combined organic extracts were washed with 1 M HCl (10 mL), water (10 mL) then brine (10 mL). The organics were dried (Na₂SO₄), filtered and concentrated. The crude residue was purified by chromatography (elution with 5% EtOAc/hexanes) to afford the title compound (66 mg) as a white solid: ¹H NMR (CDCl₃, 400 MHz) δ 7.20-7.17 (m, 4H), 4.04 (dd, 1H, J=13.7, 2.9 Hz), 3.82-3.72 (m, 2H), 3.44 (dd, 1H, J=13.7, 1.7 Hz), 2.76 (d, 1H, J=1.2 Hz), 2.69 (d, 1H, J=1.2 Hz), 1.31 (s, 3H), 0.91 (s, 3H); ¹³C NMR (CDCl₃, 100 MHz) δ 158.2, 158.0, 144.5, 143.6, 127.8, 127.6, 123.8, 123.6, 120.8, 117.9, 115.1, 112.2, 49.0, 48.8, 46.7, 45.3, 43.8, 27.6, 20.6; GCMS m/z 283 (M+).

F) 12,12-Dimethyl-10-aza-tricyclo[6.3.1.0^{2,7}]dodeca-2(7),3,5-triene hydrochloride

The title compound was prepared from 2,2,2-trifluoro-1-(12,12-dimethyl-10-aza-tricyclo[6.3.1.0^{2,7}]dodeca-2,4,6-trien-10-yl)-ethanone (21 mg, 0.074 mmol) by the method described in Example 2C to afford 11 mg of a white solid. Data for free base: ¹H NMR (CDCl₃, 400 MHz) δ 7.18-7.12 (m, 4H), 3.24 (d, 2H, J=13.3 Hz), 2.49 (d, 2H, J=13.3 Hz), 2.45 (s, 2H), 1.85 (br s, 1H), 1.30 (s, 3H), 0.81 (s, 3H); ¹³C NMR (CDCl₃, 100 MHz) δ 146.0, 126.9, 123.4, 51.5, 44.7, 29.4, 20.9; GCMS m/z 187 (M+).

### Example 24

### 12,12-Dimethyl-4-nitro-10-aza-tricyclo[6.3.1.0^{2,7}]dodeca-2(7),3,5-triene Hydrochloride

A) 2,2,2-Trifluoro-1-(12,12-dimethyl-4-nitro-10-aza-tricyclo[6.3.1.0^{2,7}]dodeca-2,4,6-trien-10-yl)-ethanone

Nitric acid (46 μL, 0.51 mmol, 69%) was slowly added to a solution of 2,2,2-trifluoro-1-(12,12-dimethyl-10-aza-tricyclo[6.3.1.0^{2,7}]dodeca-2,4,6-trien-10-yl)-ethanone (44 mg, 0.16 mmol) in TFA (0.16 mL) at 0° C. (ice bath). The mixture was allowed to warm to RT and stirred for 8 h at which time it was poured over CHCl₃ (10 mL) and water (10 mL). The solution was neutralized with sat. NaHCO₃ (aq) and partitioned. The aqueous layer was extracted with CHCl₃ (3×10 mL). The combined organics were washed with water (10 mL) then brine (10 mL) and dried (Na₂SO₄), filtered and concentrated to afford the title compound (51 mg) which was used without further purification: ¹H NMR (CDCl₃, 400 MHz) δ 8.14-8.06 (m, 2H), 7.36 (dd, 1H, J=7.9, 2.1 Hz), 4.09 (dt, 1H, J=13.7, 2.5 Hz), 3.85 (dd, 1H, J=12.8, 2.5 Hz), 3.78 (d, 1H, J=12.8 Hz), 3.48 (d, 1H, J=14.1 Hz), 2.91 (d, 1H, J=10.4 Hz), 2.84 (d, 1H, J=11.6 Hz), 1.35 (s, 3H), 0.90 (s, 3H); GCMS m/z 328 (M+).

B) 12,12-dimethyl-4-nitro-10-aza-tricyclo[6.3.1.0^{2,7}]dodeca-2(7),3,5-triene hydrochloride

The title compound was prepared from 2,2,2-trifluoro-1-(12,12-dimethyl-4-nitro-10-aza-tricyclo[6.3.1.0^{2,7}]dodeca-2,4,6-trien-10-yl)-ethanone (50 mg, 0.15 mmol) by the method described in Example 2C to afford 27 mg of a white solid. Data for free base: ¹H NMR (CDCl₃, 400 MHz) δ 8.10 (dd, 1H, J=8.3, 2.1 Hz), 8.01 (d, 1H, J=2.1 Hz), 7.29 (d, 1H, J=8.3 Hz), 3.35-3.30 (m, 2H), 2.62-2.58 (m, 4H), 1.61 (br s, 1H), 1.34 (s, 3H), 0.82 (s, 3H); ¹³C NMR (CDCl₃, 100 MHz) 154.4, 148.0, 147.6, 123.8, 123.3, 118.6, 51.6, 51.4, 45.4, 44.4, 44.3, 29.2, 20.7; GCMS m/z 232 (M+).

1. A compound of the formula 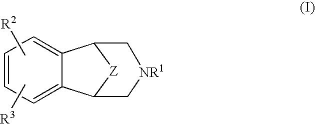
wherein Z is a group represented by the formula CR⁴R⁵ or CR⁶R⁷CR⁸R⁹;
R¹ is hydrogen, (C₁-C₆)alkyl, unconjugated (C₃-C₆)alkenyl, benzyl, XC(═O)R¹³ or —CH₂CH₂—O—(C₁-C₄)alkyl;
R² and R³ are selected, independently, from hydrogen, (C₂-C₆)alkenyl, (C₂-C₆)alkynyl, hydroxy, nitro, amino, halo, cyano, —SO_{q}(C₁-C₆)alkyl wherein q is zero, one or two, (C₁-C₆)alkylamino-, [(C₁-C₆)alkyl]₂amino-, —CO₂R¹⁰, —CONR¹¹R¹², —SO₂NR¹³R¹⁴, —C(═O)R¹⁹, —XC(═O)R¹⁹, aryl-(C₀-C₃)alkyl- or aryl-(C₀-C₃)alkyl-O—, wherein said aryl is selected from phenyl and naphthyl, heteroaryl-(C₀-C₃)alkyl- or heteroaryl-(C₀-C₃)alkyl-O—, wherein said heteroaryl is selected from five to seven membered aromatic rings containing from one to four heteroatoms selected from oxygen, nitrogen and sulfur; X²(C₀-C₆)alkyl- and X²(C₁-C₆)alkoxy-(C₀-C₆)alkyl-, wherein X² is absent or X² is (C₁-C₆)alkylamino- or [(C₁-C₆)alkyl]₂amino-, and wherein the (C₀-C₆)alkyl- or (C₁-C₆)alkoxy-(C₀-C₆)alkyl- moieties of said X²(C₀-C₆)alkyl- or X²(C₁-C₆)alkoxy-(C₀-C₆)alkyl- contains at least one carbon atom, and wherein from one to three of the carbon atoms of said (C₀-C₆)alkyl- or (C₁-C₆)alkoxy-(C₀-C₆)alkyl- moieties may optionally be replaced by an oxygen, nitrogen or sulfur atom, with the proviso that any two such heteroatoms must be separated by at least two carbon atoms, and wherein any of the alkyl moieties of said (C₀-C₆)alkyl- or (C₁-C₆)alkoxy-(C₀-C₆)alkyl- groups may be optionally substituted with from two to seven fluorine atoms, and wherein one of the carbon atoms of each of the alkyl moieties of said aryl-(C₀-C₃)alkyl- and said heteroaryl-(C₀-C₃)alkyl- may optionally be replaced by an oxygen, nitrogen or sulfur atom, and wherein each of the foregoing aryl and heteroaryl groups may optionally be substituted with one or more substituents, preferably from zero to two substituents, independently selected from (C₁-C₆)alkyl optionally substituted with from one to seven fluorine atoms, (C₁-C₆)alkoxy optionally substituted with from two to seven fluorine atoms, halo (e.g., chloro, fluoro, bromo or iodo), (C₂-C₆)alkenyl, (C₂-C₆)alkynyl, hydroxy, nitro, cyano, amino, (C₁-C₆)alkylamino-, [(C₁-C₆)alkyl]₂amino-, —CO₂R¹⁰, —CONR¹¹R¹², —SO₂NR¹³R¹⁴, —C(═O)R¹⁹ and —XC(═O)R¹⁹; with the proviso that when Z is CR⁶R⁷CR⁸R⁹ and R⁶, R⁷, R⁸ and R⁹ are all H, then R² and R³ are not both H;
or R² and R³, together with the carbons to which they are attached, form a four to seven membered monocyclic, or a ten to fourteen membered bicyclic, carbocyclic ring that can be saturated or unsaturated, wherein from one to three of the non-fused carbon atoms of said monocyclic rings, and from one to five of the carbon atoms of said bicyclic rings that are not part of the benzo ring shown in formula I, may optionally and independently be replaced by a nitrogen, oxygen or sulfur, and wherein said monocyclic and bicyclic rings may optionally be substituted with one or more substituents, preferably from zero to two substituents for the monocyclic rings and from zero to three substituents for the bicyclic rings, that are selected, independently, from (C₀-C₆)alkyl- or (C₁-C₆)alkoxy-(C₀-C₆)alkyl-, wherein the total number of carbon atoms does not exceed six and wherein any of the alkyl moieties may optionally be substituted with from one to seven fluorine atoms; nitro, oxo, cyano, halo, (C₂-C₆)alkenyl, (C₂-C₆)alkynyl, hydroxy, amino, (C₁-C₆)alkylamino-, [(C₁-C₆)alkyl]₂amino-, —CO₂R¹⁰, —CONR¹¹R¹², —SO₂NR¹³R¹⁴, —C(═O)R¹⁹, and —XC(═O)R¹⁹;
R⁴ and R⁵ are selected, independently, from H, (C₁-C₆)alkyl, F, Cl, Ph, CH₂Ph, (C₁-C₆)alkoxy, or R⁴ and R⁵, together with the carbon they are attached, form a three, four or six membered saturated ring with the proviso that R⁴ and R⁵ cannot both be H;
R⁶, R⁷, R⁸ and R⁹ are selected, independently, from H, Me, Et, Pr, Ph and CF₃;
each R¹⁰, R¹¹, R¹², R¹³, R¹⁴ and R¹⁹ is selected, independently, from hydrogen and (C₁-C₆)alkyl, or R¹¹ and R¹², or R¹³ and R¹⁴ together with the nitrogen to which they are attached, form a pyrrolidine, piperidine, morpholine, azetidine, piperazine, —N—(C₁-C₆)alkylpiperazine or thiomorpholine ring, or a thiomorpholine ring wherein the ring sulfur is replaced with a sulfoxide or sulfone; and
each X is, independently, (C₁-C₆)alkylene.

2. A compound according to claim 1, wherein said heteroaryl within the definition of R² and R³ is selected from thienyl, oxazoyl, isoxazolyl, pyridyl, pyrimidyl, thiazolyl, tetrazolyl, isothiazolyl, triazolyl, imidazolyl, tetrazolyl or pyrrolyl.

3. A compound according to claim 1, wherein said heteroaryl within the definition of R² and R³ is selected from the following: 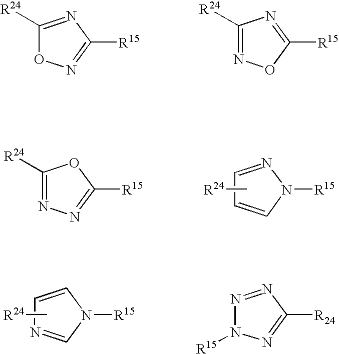
wherein one of R¹⁵ and R²⁴ is hydrogen or (C₁-C₆)alkyl, and the other is a bond to the benzo ring of formula I.

4. A compound according to claim 1, wherein R² and R³, together with the benzo ring of formula I, form a bicyclic or tricyclic ring system selected from the following: 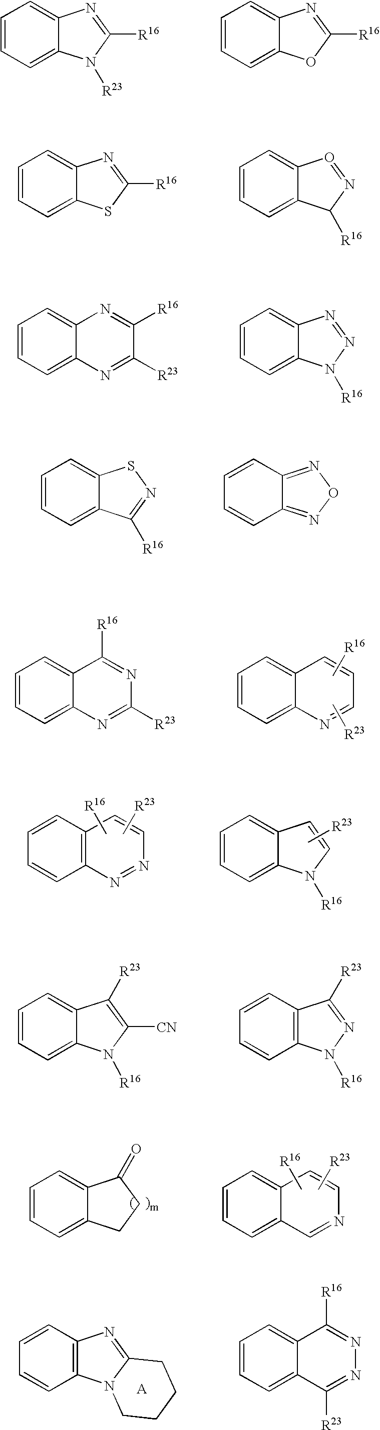
wherein R¹⁶ and R²³ are selected independently from hydrogen, (C₁-C₆)alkyl; and (C₁-C₆) alkoxy-(C₀-C₆)alkyl- wherein the total number of carbon atoms does not exceed six and wherein any of the alkyl moieties may be substituted with from one to seven fluorine atoms; nitro, cyano, halo, amino, (C₁-C₆)alkylamino-, [(C₁-C₆)alkyl]₂amino-, —CO₂R¹⁰, —CONR¹¹R¹², —SO₂NR¹³R¹⁴, —C(═O)R¹⁹, —XC(═O)R¹⁹, phenyl and monocyclic heteroaryl wherein said heteroaryl is defined as R² and R³ are defined in claim 1 and R¹⁰, R¹¹, R¹², R¹³, R¹⁴, R¹⁹ are as defined in claim 1, m is zero, one or two and wherein one of the carbon atoms of ring A may be replaced with oxygen or N(C₁-C₆)alkyl.

5. A compound according to claim 1, wherein R² and R³ do not, together with the benzo ring of formula I, form a bicyclic or tricyclic ring system.

6. A compound according to claim 1, wherein one or both of R² and R³ are —C(═O)R¹⁹ wherein R¹⁹ is (C₁-C₆)alkyl.

7. A compound according to claim 1, wherein one or both of R² and R³ are —C(═O)R¹⁹ wherein R¹⁹ is (C₁-C₆)alkyl or (C₁-C₃)alkyl which may be substituted with from one to seven fluorine atoms.

8. A compound according to claim 1, wherein one of R² and R³ is CF₃, fluoro, cyano, (C₂-C₆)alkynyl or C₂F₅.

9. A compound according to claim 1, wherein Z is CR⁴R⁵ where R⁴ is Me and R⁵ is H and the geometric structure is exo or endo.

10. A pharmaceutical composition for use in reducing nicotine addiction or aiding in the cessation or lessening of tobacco use in a mammal, comprising an amount of a compound according to claim 1 that is effective in reducing nicotine addiction or aiding in the cessation or lessening of tobacco use and a pharmaceutically acceptable carrier.

11. A method for reducing nicotine addiction or aiding in the cessation or lessening of tobacco use in a mammal, comprising administering to said mammal an amount of a compound according to claim 1 that is effective in reducing nicotine addiction or aiding in the cessation or lessening of tobacco use.

12. A pharmaceutical composition for treating a disorder or condition selected from inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrythmias, gastric acid hypersecretion, ulcers, pheochromocytoma, progressive supranuclear palsy, chemical dependencies and addictions, dependencies on, or addictions to nicotine (or tobacco products), alcohol, benzodiazepines, barbiturates, opioids or cocaine, headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, petit mal absence epilepsy, senile dementia of the Alzheimer's type (AD), Parkinson's disease (PD), attention deficit hyperactivity disorder (ADHD), attention deficit disorder (ADD), restless legs syndrome (RLS), mild cognitive impairment, cognitive enhancement in schizophrenia, drug induced extrapyramidal symptoms, conduct disorder, oppositional defined disorder, anxiety in anxious smokers, cardiovascular risk in pregnancy, delayed ejaculation, emesis, symptoms due to injury inflicted by biological warfare, diarrhea, nicotine gum addiction, sleep prevention, ischemia, and Tourette's Syndrome in a mammal, comprising an amount of a compound according to claim 1 that is effective in treating such disorder or condition and a pharmaceutically acceptable carrier.

13. A method for treating a disorder or condition selected from inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrythmias, gastric acid hypersecretion, ulcers, pheochromocytoma, progressive supranuclear palsy, chemical dependencies and addictions, dependencies on, or addictions to nicotine (or tobacco products), alcohol, benzodiazepines, barbiturates, opioids or cocaine, headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, petit mal absence epilepsy, senile dementia of the Alzheimer's type (AD), Parkinson's disease (PD), attention deficit hyperactivity disorder (ADHD), attention deficit disorder (ADD), restless legs syndrome (RLS), mild cognitive impairment, cognitive enhancement in schizophrenia, drug induced extrapyramidal symptoms, conduct disorder, oppositional defined disorder, anxiety in anxious smokers, cardiovascular risk in pregnancy, delayed ejaculation, emesis, symptoms due to injury inflicted by biological warfare, diarrhea, nicotine gum addiction, sleep prevention, ischemia, and Tourette's Syndrome in a mammal, comprising administering to a mammal in need of such treatment an amount of a compound according to claim 1 that is effective in treating such disorder or condition.

14. A compound of the formula 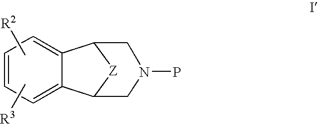
wherein R² and R³ are defined as in claim 1; and P is COOR¹⁷ wherein R¹⁷ is allyl, 2,2,2-trichloroethyl or (C₁-C₆)alkyl; —C(═O)NR¹⁰R¹¹ wherein R¹¹ and R¹² are defined as in claim 1; —C(═O)H; —C(═O)(C₁-C₆)alkyl or —C(═S)(C₁-C₆)alkyl wherein the alkyl moiety may optionally be substituted with from 1 to 3 halo atoms, preferably with from 1 to 3 fluoro or chloro atoms; benzyl, or t-butoxycarbonyl.

15. A method for reducing nicotine addiction or aiding in the cessation or lessening of tobacco use in a mammal, comprising administering to said mammal an amount of a compound comprising an amount of a compound of the formula 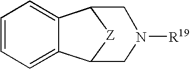
wherein Z is as defined above and R¹⁹ is selected from the group consisting of hydrogen or (C₁-C₆)alkyl, or a pharmaceutically acceptable salt thereof, that is effective in reducing nicotine addiction or aiding in the cessation or lessening of tobacco use.

16. A method for treating a disorder or condition selected from inflammatory bowel disease, ulcerative colitis, pyoderma gangrenosum, Crohn's disease, irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrythmias, gastric acid hypersecretion, ulcers, pheochromocytoma, progressive supranuclear palsy, chemical dependencies and addictions, dependencies on, or addictions to nicotine (or tobacco products), alcohol, benzodiazepines, barbiturates, opioids or cocaine, headache, migraine, stroke, traumatic brain injury (TBI), obsessive-compulsive disorder (OCD), psychosis, Huntington's chorea, tardive dyskinesia, hyperkinesia, dyslexia, schizophrenia, multi-infarct dementia, age-related cognitive decline, epilepsy, petit mal absence epilepsy, senile dementia of the Alzheimer's type (AD), Parkinson's disease (PD), attention deficit hyperactivity disorder (ADHD), attention deficit disorder (ADD), restless legs syndrome (RLS), mild cognitive impairment, cognitive enhancement in schizophrenia, drug induced extrapyramidal symptoms, conduct disorder, oppositional defined disorder, anxiety in anxious smokers, cardiovascular risk in pregnancy, delayed ejaculation, emesis, symptoms due to injury inflicted by biological warfare, diarrhea, nicotine gum addiction, sleep prevention, ischemia, and Tourette's Syndrome in a mammal, comprising administering to a mammal in need of such treatment an amount of a compound of the formula 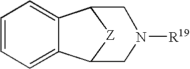
wherein Z is as defined above and R¹⁹ is selected from the group consisting of hydrogen or (C₁-C₆)alkyl, or a pharmaceutically acceptable salt thereof, that is effective in treating such disorder or condition.

17. A compound according to claim 1 selected from the group consisting of:
10-aza-tricyclo[6.3.2.0^{2,7}]trideca-2(7),3,5-triene;
4-nitro-10-aza-tricyclo[6.3.2.0^{2,7}]trideca-2(7),3,5-triene;
6-methyl-5-thia-7,13-diazatetracyclo[9.3.2.0^{2,10}.0^{4,8}]hexadeca-2(10),3,6,8-tetraene;
5,14-diazatetracyclo[10.3.2.0^{2,11}.0^{4,9}]heptadeca-2(11),3,5,7,9-pentaene;
6-methyl-5,14-diazatetracyclo[10.3.2.0^{2,11}.0^{4,9}]heptadeca-2(11),3,5,7,9-pentaene;
4-fluoro-10-aza-tricyclo[6.3.2.0^{2,7}]trideca-2,4,6-triene;
4-chloro-10-azatricyclo[6.3.2.0^{2,7}]trideca-2,4,6-triene;
4-bromo-10-azatricyclo[6.3.2.0^{2,7}]trideca-2,4,6-triene;
10-aza-tricyclo[6.3.2.0^{2,7}]trideca-2,4,6-triene-4-carbonitrile;
1-(10-azatricyclo[6.3.2.0^{2,7}]trideca-2(7),3,5-trien-4-yl)-1-ethanone;
4,5-dinitro-10-aza-tricyclo[6.3.2.0^{2,7}]trideca-2(7),3,5-triene;
5,8,14-triazatetracyclo[10.3.2.0^{2,11}.0^{4,9}]-heptadeca-2(11),3,5,7,9-pentaene;
5,7,13-triazatetracyclo[9.3.2.0^{2,10}.0^{4,8}]-hexadeca-2(10),3,5,8-tetraene;
7-methyl-5,7,13-triazatetracyclo[9.3.2.0^{2,10}.0^{4,8}]-hexadeca-2(10),3,5,8-tetraene;
7-ethyl-5,7,13-triazatetracyclo[9.3.2.0^{2,10}.0^{4,8}]-hexadeca-2(10),3,5,8-tetraene;
7-propyl-5,7,13-triazatetracyclo[9.3.2.0^{2,10}.0^{4,8}]-hexadeca-2(10),3,5,8-tetraene;
6-methyl-5,7,13-triazatetracyclo[9.3.2.0^{2,10}.0^{4,8}]-hexadeca-2(10),3,5,8-tetraene;
6,7-dimethyl-5,7,13-triazatetracyclo[9.3.2.0^{2,10}.0^{4,8}]-hexadeca-2(10),3,5,8-tetraene;
6-methyl-7-ethyl-5,7,13-triazatetracyclo[9.3.2.0^{2,10}.0^{4,8}]-hexadeca-2(10),3,5,8-tetraene;
6-methyl-7-propyl-5,7,13-triazatetracyclo[9.3.2.0^{2,10}.0^{4,8}]-hexadeca-2(10),3,5,8-tetraene;
6-methyl-5-oxa-7,13-diazatetracyclo[9.3.2.0^{2,10}.0^{4,8}]-hexadeca-2(10),3,6,8-tetraene;
12-exo-methyl-4-nitro-10-aza-tricyclo[6.3.1.0^{2,7}]dodeca-2(7),3,5-triene;
12,12-dimethyl-10-aza-tricyclo[6.3.1.0^{2,7}]dodeca-2(7),3,5-triene; and
12,12-dimethyl-4-nitro-10-aza-tricyclo[6.3.1.0^{2,7}]dodeca-2(7),3,5-triene;
and pharmaceutically acceptable salts thereof.

18. A compound according to claim 13 selected from the group consisting of:
N-(10-trifluoroacetyl-10-aza-tricyclo[6.3.2.0^{2,7}]trideca-2(7),3,5-trien-4-yl)-acetamide;
2,2,2-trifluoro-N-(10-trifluorothioacetyl-10-aza-tricyclo[6.3.2.0^{2,7}]trideca-2(7),3,5-trien-4-yl)-thioacetamide;
1-(4-amino-10-aza-tricyclo[6.3.2.0^{2,7}]trideca-2(7),3,5-trien-10-yl)-2,2,2-trifluoro-ethanone;
1-(6-methyl-5,14-diazatetracyclo[10.3.2.0^{2,11}.0^{4,9}]heptadeca-2(11),3,5,7,9-pentaene)-2,2,2-trifluoro-ethanone;
1-(4-fluoro-10-aza-tricyclo[6.3.2.0^{2,7}]trideca-2(7),3,5-trien-10-yl)-2,2,2-trifluoro-ethanone;
1-(4-chloro-10-aza-tricyclo[6.3.2.0^{2,7}]trideca-2(7),3,5-trien-10-yl)-2,2,2-trifluoro-ethanone;
1-(4-amino-10-aza-tricyclo[6.3.2.0^{2,7}]trideca-2(7),3,5-trien-10-yl)-2,2,2-trifluoro-ethanone;
4-iodo-10-aza-tricyclo[6.3.2.0^{2,7}]trideca-2(7),3,5-triene-10-carboxylic acid tert-butyl ester;
4-cyano-10-aza-tricyclo[6.3.2.0^{2,7}]trideca-2(7),3,5-triene-10-carboxylic acid tert-butyl ester;
1-(4-acetyl-10-aza-tricyclo[6.3.2.0^{2,7}]trideca-2(7),3,5-trien-10-yl)-2,2,2-trifluoro-ethanone;
1-(4,5-dinitro-10-aza-tricyclo[6.3.2.0^{2,7}]trideca-2(7),3,5-trien-10-yl)-2,2,2-trifluoro-ethanone;
1-(4,5-diamino-10-aza-tricyclo[6.3.2.0^{2,7}]trideca-2(7),3,5-trien-10-yl)-2,2,2-trifluoro-ethanone;
1-(5,8,14-triazatetracyclo[10.3.2.0^{2,11}.0^{4,9}]heptadeca-2(11),3,5,7,9-pentaene)-2,2,2-trifluoro-ethanone;
1-(5,7,13-triazatetracyclo[9.3.2.0^{2,10}.0^{4,8}]hexadeca-2(10),3,5,8-tetraene)-2,2,2-trifluoro-ethanone;
5,7,13-triazatetracyclo[9.3.2.0^{2,10}.0^{4,8}]hexadeca-2(10),3,5,8-tetraene-13-carboxylic acid tert-butyl ester;
7-methyl-5,7,13-triazatetracyclo[9.3.2.0^{2,10}.0^{4,8}]hexadeca-2(10),3,5,8-tetraene-13-carboxylic acid tert-butyl ester;
7-ethyl-5,7,13-triazatetracyclo[9.3.2.0^{2,10}.0^{4,8}]hexadeca-2(10),3,5,8-tetraene-13-carboxylic acid tert-butyl ester;
7-propyl-5,7,13-triazatetracyclo[9.3.2.0^{2,10}.0^{4,8}]hexadeca-2(10),3,5,8-tetraene-13-carboxylic acid tert-butyl ester;
1-(6-methyl-5,7,13-triazatetracyclo[9.3.2.0^{2,10}.0^{4,8}]hexadeca-2(10),3,5,8-tetraene)-2,2,2-trifluoro-ethanone;
6-methyl-5,7,13-triazatetracyclo[9.3.2.0^{2,10}.0^{4,8}]hexadeca-2(10),3,5,8-tetraene-13-carboxylic acid tert-butyl ester;
6,7-dimethyl-5,7,13-triazatetracyclo[9.3.2.0^{2,10}.0^{4,8}]hexadeca-2(10),3,5,8-tetraene-13-carboxylic acid tert-butyl ester;
6-methyl-7-ethyl-5,7,13-triazatetracyclo[9.3.2.0^{2,10}.0^{4,8}]hexadeca-2(10),3,5,8-tetraene-13-carboxylic acid tert-butyl ester;
6-methyl-7-propyl-5,7,13-triazatetracyclo[9.3.2.0^{2,10}.0^{4,8}]hexadeca-2(10),3,5,8-tetraene-13-carboxylic acid tert-butyl ester;
2,2,2-trifluoro-1-(4-hydroxy-5-nitro-10-aza-tricyclo[6.3.2.0^{2,7}]trideca-2(7),3,5-trien-10-yl)-ethanone;
2,2,2-trifluoro-1-(4-hydroxy-5-amino-10-aza-tricyclo[6.3.2.0^{2,7}]trideca-2(7),3,5-trien-10-yl)-ethanone;
2,2,2-trifluoro-1-(6-methyl-5-oxa-7,13-diazatetracyclo[9.3.2.0^{2,10}.0^{4,8}]hexadeca-2(10),3,6,8-tetraene)-ethanone;
2,2,2-trifluoro-1-(12-exo-methyl-10-aza-tricyclo[6.3.1.0^{2,7}]dodeca-2,4,6-trien-10-yl)-ethanone;
2,2,2-trifluoro-1-(12-exo-methyl-4-nitro-10-aza-tricyclo[6.3.1.0^{2,7}]dodeca-2,4,6-trien-10-yl)-ethanone;
2,2,2-trifluoro-1-(12,12-dimethyl-10-aza-tricyclo[6.3.1.0^{2,7}]dodeca-2,4,6-trien-10-yl)-ethanone; and
2,2,2-trifluoro-1-(12,12-dimethyl-4-nitro-10-aza-tricyclo[6.3.1.0^{2,7}]dodeca-2,4,6-trien-10-yl)-ethanone;
and pharmaceutically acceptable salts thereof.
